(19) United States
(12) Patent Application Publication
(10) Pub. No.: US 2022/0249002 A1
(43) Pub. Date: Aug. 11, 2022

(54) BIOSENSOR EXCITATION METHODS, AND ASSOCIATED SYSTEMS, DEVICES, AND METHODS

(71) Applicant: INFORMED DATA SYSTEMS INC. d/b/a ONE DROP, New York, NY (US)

(72) Inventors: Matthew Chapman, Oakland, CA (US); Ashwin Pushpala, San Francisco, CA (US); Ezekial Carnes-Mason, San Francisco, CA (US); Jason Goldberg, San Francisco, CA (US)

(21) Appl. No.: 17/667,530

(22) Filed: Feb. 8, 2022

Related U.S. Application Data
(60) Provisional application No. 63/147,206, filed on Feb. 8, 2021.

(51) Int. Cl. A61B 5/279; A61B 5/256; A61B 5/145

(52) U.S. Cl. CPC A61B 5/279; A61B 5/14532; A61B 5/256

(57) ABSTRACT

Systems and methods for operating a biosensor are disclosed herein. In some embodiments, the biosensor is configured to access a user's interstitial fluid to determine the concentration of one or more analytes of interest. The method can include applying an excitation voltage to a working electrode of biosensor. The excitation voltage can be a time-varying waveform, such as a square waveform. The method can further include measuring a signal generated by the biosensor and analyzing the signal to determine one or more parameters of the biosensor and/or surrounding environment.

### CROSS-REFERENCE TO RELATED APPLICATION

The present application claims priority to U.S. Provisional Patent Application No. 63/147,206, filed Feb. 8, 2021, the disclosure of which is incorporated by reference herein in its entirety.

### TECHNICAL FIELD

The present disclosure relates generally to personalized healthcare and, in particular, to systems and methods for biomonitoring and healthcare guidance. For example, several embodiments of the present technology are directed (a) to applying a perturbation to a biosensor excitation voltage and/or (b) to analyzing a resulting response using a model to determine effective surface area of an electrode, an absolute concentration of an analyte in a fluid sample, and/or other parameters of interest.

### BACKGROUND

Many individuals suffer from chronic health conditions, such as diabetes, pre-diabetes, hypertension, hyperlipidemia, and the like. For example, diabetes mellitus (DM) is a group of metabolic disorders characterized by high blood glucose levels over a prolonged period. Typical symptoms of such conditions include frequent urination, increased thirst, increased hunger, etc. If left untreated, diabetes can cause many complications. There are three main types of diabetes: Type 1 diabetes, Type 2 diabetes, and gestational diabetes. Type 1 diabetes results from the pancreas' failure to produce enough insulin. In Type 2 diabetes, cells fail to respond to insulin properly. Gestational diabetes occurs when pregnant women without a previous history of diabetes develop high blood glucose levels.

Diabetes affects a significant percentage of the world's population. Timely and proper diagnoses and treatment are essential to maintaining a relatively healthy lifestyle for individuals with diabetes. Application of treatment typically relies on accurate determination of glucose concentration in the blood of an individual at a present time and/or in the future. However, conventional blood glucose monitoring systems may be unable to provide real-time analytics, personalized analytics, or blood glucose concentration forecasting, or may not provide such information in a rapid, reliable, and accurate manner. Thus, there is a need for improved systems and methods for biomonitoring and/or providing personalized healthcare recommendations or information for the treatment of diabetes and other chronic conditions.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments depicted, but are for explanation and understanding only.

FIG. 1 is a partially schematic diagram of a computing environment in which a biomonitoring and healthcare guidance system operates in accordance with various embodiments of the present technology.

FIGS. 2A and 2B are partially schematic illustrations of biosensor devices configured in accordance with various embodiments of the present technology.

FIGS. 3A-3E are partially schematic illustrations of a biosensor patch device configured in accordance with various embodiments of the present technology.

FIG. 4 is a flow diagram illustrating a method of operating biosensor devices in accordance with various embodiments of the present technology.

FIG. 5 illustrates line plots of example excitation signal perturbations used in various embodiments of the present technology.

FIG. 6A illustrates a line plot of an excitation signal including a voltage step perturbation used in various embodiments of the present technology.

FIG. 6B illustrates line plots of a current response and a charge response of a biosensor to the voltage step perturbation of FIG. 6A in accordance with various embodiments of the present technology.

FIG. 7 illustrates (a) a line plot of an expected current response of a biosensor assuming infinite bandwidth and (b) line plots of current responses of the biosensor to various low pass cutoffs, in accordance with various embodiments of the present technology.

FIG. 8A is a line plot of current responses of a biosensor to perturbations in an excitation signal applied to the biosensor in accordance with various embodiments of the present technology.

FIG. 8B is a line plot of capacitive charging component signals and diffusion limited component signals of the current responses of FIG. 8A, as separated in accordance with various embodiments of the present technology.

FIG. 9 is a flow diagram illustrating another method of operating biosensor devices in accordance with various embodiments of the present technology.

### DETAILED DESCRIPTION

The present technology generally relates to systems and methods for biomonitoring and providing personalized healthcare. For example, several embodiments of the present technology are directed (a) to modeling an expected response of biosensor to a perturbation (e.g., a rising step change) in an excitation signal applied to the biosensor; (b) to monitoring an actual response of the biosensor to the perturbation; and (c) to deconvoluting system-dependent parameters and/or properties of the environment (e.g., tissue) surrounding the biosensor from concentration of an analyte of interest in a body fluid of a user. In turn, the system-dependent parameters and/or the properties of the surrounding environment can be used to determine an extent or quality of application of the biosensor to the user's body, to calibrate or inform various operations of the biomonitoring system, and/or to determine other information of interest (e.g., absolute concentrations of the analyte of interest in the body fluid, extent of skin healing proximate the biosensor, and/or hydration level of the user).

In some embodiments, a biomonitoring and healthcare guidance system is configured to generate personalized self-care recommendations (e.g., recommendations relating to sleep, exercise, diet, etc.) to guide a patient in effectively managing and/or improving a chronic condition (e.g., diabetes, pre-diabetes, hypertension, hyperlipidemia, etc.). The system can continuously or periodically update and/or adapt the self-care recommendations, for example, based on data from the particular patient as well as data from a plurality of other patients. The system can guide individuals toward self-care changes that are likely to improve their chronic health conditions, support them in making those changes, and/or adapt or update continuously over time.

As discussed in greater detail below, a sensing element (e.g., an electrode, a microneedle, etc.) of a biosensor can be positioned to access a body fluid of a user within or beneath the user's skin. For example, signals output from an electrode can provide indications of concentrations of one or more analytes of interest in the body fluid. The output signals can depend at least in part on an amount of the electrode that accesses and/or interacts with the body fluid. A functionalized detection-surface parameter of the electrode can be determined based on the detected response(s) to perturbation signals. The functionalized detection-surface parameter can be the amount of the surface of the electrode (a) that is configured to detect an analyte of interest in a body fluid, (b) that accesses/interacts with the body fluid (e.g., due to positioning of the electrode at a depth within or beneath the user's skin), and/or (c) that contributes to signals output from the electrode that provide indications of concentrations of one or more analytes of interest in the body fluid.

In some embodiments, biomonitoring systems of the present technology are configured to apply excitation signals that include one or more electrode interrogation signals. The electrode interrogation signals can include, for example, diffusion-inducing signals, transient response signals (e.g., transient diffusion signals), perturbation signals (e.g., signals with rising step changes), or the like. Output from the electrodes in response to the electrode interrogation signals can be used to determine one or more operational parameters (e.g., system-dependent parameters, properties of the interstitial fluid surrounding the electrode, one or more properties of tissue surrounding the electrode, or any combination thereof). The one or more operational parameters can be used to control operation of the biomonitoring systems to enhance performance. In some embodiments, the biomonitoring systems performs a routine to determine functionalized detection-surface parameters for individual electrodes, sets of electrodes, or the like. The functionalized detection-surface parameters can be determined periodically or continuously based on user settings. The functionalized detection-surface parameters can be used to generate excitation signals, signal processing routines, signal filter routines, signal correction routines, or the like. The characteristics of the electrode interrogation signals (e.g., amplitude, waveform, duration, number, etc.) can be selected based on, for example, electrode characteristics (e.g., size, chemistry, etc.), tissue characteristics, body fluid characteristics, or the like.

Embodiments of the present disclosure will be described more fully hereinafter with reference to the accompanying drawings, and in which example embodiments are shown. Embodiments of the claims may, however, be embodied in many different forms and should not be construed as limited to the embodiments set forth herein. The examples set forth herein are non-limiting examples and are merely examples among other possible examples. In addition, the headings provided herein are for convenience only and do not interpret the scope or meaning of the claimed present technology.

### Overview

Biomonitoring systems can include biosensors that are configured to sense one or more analytes in a body fluid by employing various techniques, such as electrochemical sensing techniques or other suitable techniques. In a specific example, a biomonitoring system can include a biosensor having a plurality of sensing elements, such as electrodes (e.g., working electrode, a reference electrode, and/or a counter electrode). The electrodes can be inserted into a user's body within or below the user's skin to access a body fluid (e.g., interstitial fluid, blood), and the biomonitoring system can apply an excitation signal (e.g., a voltage) to a working electrode that differs from a signal (e.g., another voltage) applied to a corresponding reference electrode, for example, such that a difference in potential is created between the working electrode and the reference electrode. In turn, the electrodes can produce and output signals (e.g., voltages, currents, resistances, capacitances, impedances, gravimetric signals, and/or various other suitable signals) that are affected by—and therefore provide an indication of—concentrations of one or more analytes of interest in the body fluid. Based at least in part on these signals, the biomonitoring system can determine the concentrations of the one or more analytes of interest in the body fluid, and the concentration information can be used, at least in part, to provide an indication of various health conditions of the user.

The excitation signal applied to a working electrode of a biosensor is often applied using constant, direct current (DC) signal. As such, the biosensor commonly operates in a diffusion limited steady state in which diffusion limited current observed at the working electrode depends on various factors, including a surface area of the working electrode, a diffusion coefficient of an analyte of interest, and a concentration gradient of the analyte of interest at the surface of the working electrode. Thus, in order to calculate an absolute concentration of the analyte of interest, several properties of the biomonitoring system (e.g., the surface area, functionalized detection-surface parameter, diffusion properties) must either be assumed or measured. But these properties often are difficult to measure or isolate from analyte concentrations when the biosensor operates in the diffusion limited steady state. In addition, these properties often depend on factors (e.g., manufacturing variability, extent of proper application) that can make assumptions regarding these properties difficult and/or largely inaccurate.

For example, consider a first working electrode of a biosensor having a first surface area, and a second working electrode of the same or another biosensor having a second surface area. The first surface area of the first working electrode may differ from the second surface area of the second working electrode simply due to manufacturing variability, rendering if difficult to make accurate assumptions of the surface area of a working electrode based on manufacturing specifications alone. Additionally, or alternatively, if 100% of the first surface area of the first working electrode is available to detect an analyte of interest in a body fluid but only 90% accesses the body fluid when a user applies the first biosensor to his/her body, the effective surface area or the functionalized detection-surface parameter of the first working electrode is reduced by 10% in comparison to the first surface area. In turn, this 10% reduction will affect the diffusion limited current observed at the first working electrode but will be difficult to quantity and/or separate from an analyte concentration contribution to the diffusion limited current. Continuing with the above example, when the user applies the second working electrode to his/her body, the effective surface area or the functionalized detection-surface parameter of the second working electrode can be reduced by another percentage (e.g., greater than or less than 10%) in comparison to the second surface area and/or can differ from the effective surface area or the functionalized detection-surface parameter of the first working electrode described above. In other words, the effective surface area or the functionalized detection-surface parameter of working electrodes of biomonitoring systems can be largely unique to each use or application, and/or can differ across biomonitoring systems and/or biosensors.

To address these concerns, the biomonitoring systems of the present technology are configured to apply excitation signals (e.g., drive signals, interrogation signals, etc.) that include one or more perturbations (e.g., rising step changes or another time-varying characteristic) to electrodes (e.g., working electrodes) of biosensors. In particular, several embodiments of the present technology (a) model a biosensor response (e.g., a transient response, an expected current response) to a perturbation in an excitation signal applied to an electrode of the biosensor, (b) apply the perturbation to the electrode to induce a change in the electrode's diffusion limited steady state (or to move the electrode out of the diffusion limited steady state), and (c) monitor an actual response (e.g., an actual transient response, an actual current response) of the biosensor to the perturbation. In turn, the model and the actual response can be used to deconvolute various contributions (e.g., capacitive charging contributions, diffusion limited contributions, adsorbed species contributions) and to determine various parameters/properties of the biomonitoring system (e.g., effective surface area or functionalized detection-surface parameter of the electrode, diffusion properties) and/or of the surrounding environment (e.g., diffusion properties, body fluid resistance).

In some embodiments, parameters/properties determined using the actual response and the model can be used to determine additional related parameters of the biomonitoring system and/or the surrounding environment. For example, the effective surface area or the functionalized detection-surface parameter of the working electrode can be used to (a) detect application of the biomonitoring system (e.g., of the biosensor) to a user's body and/or access to a body fluid within or below the user's skin; (b) determine an extent or quality of application, such as whether the biomonitoring system was applied correctly; (c) determine whether to instruct the user to reapply the biomonitoring system (e.g., when the extent or quality of application is not sufficient to accurately measure or monitor concentrations of one or more analytes of interest in a body fluid); and/or (d) inform various operations of the biomonitoring system, such as calibration routines, application of correction factors, and/or adjustment of drive signals. As another example, the effective surface area and/or a diffusion coefficient of an analyte of interest can be used to determine absolute concentrations of the analyte of interest in the body fluid (e.g., while the biosensor operates in the diffusion limited steady state).

In these and other embodiments, the biomonitoring systems can monitor determined parameters/properties over time using the model and actual responses of the biosensor to perturbations. For example, before a biosensor is applied to a user's body, a membrane (e.g., a selective transport membrane of a needle or microneedles corresponding to an electrode, such as a working electrode) of the biosensor that is positioned at or proximate one or more electrodes of the biosensor and/or that is configured to interact with one or more analytes of interest in a body fluid can be dry. When the biosensor is applied to the user's body such that the membrane accesses the body fluid, the membrane begins to hydrate. As the membrane hydrates, diffusion properties of the membrane can change. Diffusion properties of the membrane can also change depending at least in part on potential hydrogen (pH) of analyte concentrations in the body fluid and/or biofouling, either of which can be unique to or dependent upon the user's physiology. Therefore, the biomonitoring system can use the model and actual responses of the biosensor to perturbations to determine and monitor changes of the diffusion properties of the membrane over time, and/or to inform various operations (e.g., calibration routines) or other parameters (e.g., correction factors) of the biomonitoring system. As another example, the biomonitoring systems of the present technology can monitor diffusion properties of the biomonitoring systems overtime as a proxy to determine the extent of healing (e.g., of the user's skin) proximate the electrodes of the biosensors.

Biomonitoring systems of the present technology therefore offer several advantages over other systems and devices. For example, biomonitoring systems of the present technology are expected to detect and/or determine the extent or quality of application of the biomonitoring systems to a user's body. In particular, biomonitoring systems of the present technology are expected to determine whether the biomonitoring systems have been properly applied and/or are expected to determine appropriate correction factors to account for variations or differences in one or more system parameters (e.g., the effective surface area or functionalized detection-surface parameter of an electrode or diffusion properties of a biosensor), such as across applications, across users, across biomonitoring systems, across biosensors, across electrodes of a biosensor, and/or in comparison to optimal, ideal, or baseline system parameters. As such, biomonitoring systems of the present technology are expected to reduce, minimize, eliminate errors that can occur due to improper or incomplete application of the biomonitoring systems to a user's body. As another example, biomonitoring systems of the present technology are expected to more accurately determine concentrations of analytes in a body fluid, such as absolute concentrations of analytes of interest in the body fluid, for example, while the biomonitoring systems operate in a diffusion limited steady state. As still another example, biomonitoring systems of the pre sent technology are expected to more appropriately account for changes in one or more system parameters/properties over time and/or in the surrounding environment. More specifically, biomonitoring systems of the present technology can monitor one or more parameters/properties (e.g., functionalized detection-surface parameter of electrodes, diffusion properties) over time and can apply appropriate correction factors to the biosensor and/or issue appropriate notifications or alerts (e.g., to the user) based at least in part on detected changes in the system parameters.

### Systems for Biomonitoring and Healthcare Guidance

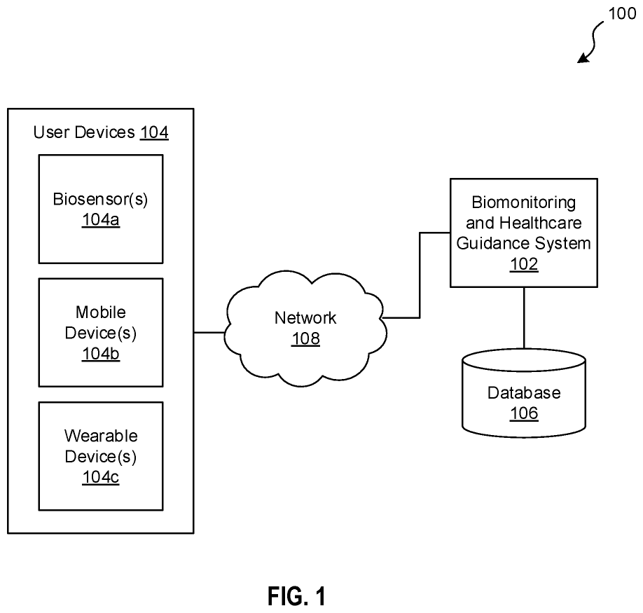

FIG. 1 is a schematic diagram of a computing environment 100 in which a biomonitoring and healthcare guidance system 102 (“system 102”) operates, in accordance with various embodiments of the present technology. As shown in FIG. 1, the system 102 is operably coupled to one or more user devices 104 via a network 108. The system 102 is also operably coupled to at least one database or storage component 106 (“database 106”). The system 102 can include processors, memory, and/or other software and/or hardware components configured to implement the various methods described herein. For example, the system 102 can be configured to monitor a user's health state and provide information to support personalized healthcare, as described in greater detail below.

The health state can be any status, condition, parameter, etc. that is associated with or otherwise related to the user's health. In some embodiments, the system 102 receives input data and performs monitoring, processing, analysis, forecasting, interpretation, etc. of the input data in order to generate instructions, notifications, recommendations, support, and/or other information to the user that may be useful for self-care of diseases or conditions, such as chronic conditions (e.g., diabetes (type 1 and type 2), pre-diabetes, hypertension, hyperlipidemia, etc.), acute conditions, etc. For example, the system 102 can be used to identify, manage, and/or monitor a variety of different diseases, conditions, and/or other health states, including, but not limited to: diabetes and associated conditions (e.g., hypoglycemia, hyperglycemia, ketoacidosis), liver diseases (e.g., hepatitis A, hepatitis B, hepatitis C, fatty liver disease, cirrhosis, live failure), cardiovascular diseases (e.g., congestive heart failure, coronary artery disease, peripheral vascular disease, hypertension, arrhythmia, cardiomyopathy), cancer (e.g., bladder cancer, breast cancer, colorectal cancer, endometrial cancer, kidney cancer, leukemia, liver cancer, lung cancer, skin cancer, lymphoma, pancreatic cancer, prostate cancer, thyroid cancer), lung diseases (e.g., asthma, chronic obstructive pulmonary disease, hypoxia, bronchitis, cystic fibrosis), kidney diseases (e.g., chronic kidney disease), brain conditions (e.g., acute brain conditions, chronic brain conditions), ophthalmological diseases, intoxication, dehydration, hyponatremia, shock, heat stroke, infection, sepsis, trauma, water retention, bleeding, endocrine disorders, muscle breakdown, malnutrition, body function (e.g., lung functions, heart functions, kidney functions, thyroid functions, adrenal functions, etc.), women's health (e.g., gynecological diseases and conditions such as polycystic ovary syndrome (PCOS), pregnancy, fertility), drug use (e.g., smoking, alcohol, or other drugs), physical performance (e.g., athletic performance), anaerobic activity, weight loss or gain, obesity, nutrition, eating disorders, metabolism (e.g., lipid metabolism, protein metabolism, aerobic metabolism), wellness, mental health, focus, stress, effects of medication, medication levels, health indicators, and/or user compliance. For example, the system 102 can be used to diagnose, monitor, analyze, track, forecast, interpret, and/or provide digital therapy using behavior change, drug or therapy titration, risk assessment, or the like.

The input data for the system 102 can include health-related information, contextual information, and/or any other information relevant to the user's health state. For example, health-related information can include levels or concentrations of a biomarker, such as glucose, electrolytes (e.g., bicarbonate, potassium, sodium, magnesium, chloride, lactic acid), neurotransmitters, amino acids, hormones, alcohols, gases (e.g. oxygen, carbon dioxide, etc.), creatinine, blood urea nitrogen (BUN), ketones, cholesterol, triglycerides, disease biomarkers (e.g., cancer biomarkers, cardiovascular disease biomarkers), lactic acid, drugs, pH, cell count, and/or other biomarkers. Health-related information can also include physiological and/or behavioral parameters, such as vitals (e.g., heart rate, body temperature (such as skin temperature), blood pressure (such as systolic and/or diastolic blood pressure), respiratory rate), cardiovascular data (e.g., pacemaker data, arrhythmia data), body function data, meal or nutrition data (e.g., number of meals; timing of meals; number of calories; amount of carbohydrates, fats, sugars, etc.), physical activity or exercise data (e.g., time and/or duration of activity; activity type such as walking, running, swimming; strenuousness of the activity such as low, moderate, high; etc.), sleep data (e.g., number of hours of sleep, average hours of sleep, variability of hours of sleep, sleep-wake cycle data, data related to sleep apnea events, sleep fragmentation (such as fraction of nighttime hours awake between sleep episodes, etc.)), stress level data (e.g., cortisol and/or other chemical indicators of stress levels, perspiration), a1c data, etc. Health-related information can also include medical history data (e.g., weight, age, sleeping patterns, medical conditions, cholesterol levels, triglyceride levels, disease type, family history, user health history, diagnoses, tobacco usage, alcohol usage, etc.), diagnostic data (e.g., molecular diagnostics, imaging), medication data (e.g., timing and/or dosages of medications such as insulin), personal data (e.g., name, gender, demographics, social network information, etc.), and/or any other data, and/or any combination thereof. Contextual information can include user location (e.g., GPS coordinates, elevation data), environmental conditions (e.g., air pressure, humidity, temperature, air quality, etc.), and/or combinations thereof.

Table 1 below lists examples of health parameters and associated diseases, conditions, and/or health states. The systems and devices described herein can be configured to monitor any of the health parameters listed in Table 1.

TABLE 1
Representative Health Parameters for Biomonitoring

| Health Parameter | Disease/Condition/Health State |
|---|---|
| Glucose | Diabetes, Weight Loss, Athletic Performance, |
| | Nutrition, Wellness, Focus |
| Oxygen | Hypoxia, Athletic Performance, Cardiac Health, |
| | Lung Function |
| Potassium | Dehydration, Cardiac Health, Diabetes, Kidney |
| | Disease, Blood Pressure |
| Sodium | Dehydration, Acute/Chronic Brain Conditions, |
| | Lung Function, Liver Function, Cardiac Health, |
| | Kidney Health, Thyroid, Adrenal |
| Lactic Acid | Shock, Sepsis, Anaerobic Activity, Metabolism, |
| | Liver Failure, Diabetic Ketoacidosis, Drugs/Toxins, |
| | Kidney Disease, Hypoxia |
| Urea/BUN | Kidney Disease, Sepsis, Hypoxia, Protein |
| | Metabolism, Nutrition |
| Ketones | Diabetic Ketoacidosis, Nutrition, Weight Loss, |
| | Lipid Metabolism |
| Bicarbonate | Kidney Disease, Liver Disease, Lung Disorders, |
| | Blood Pressure, Aerobic Metabolism |
| Temperature | Infection, Fertility, Metabolism, Athletic |
| | Performance, Heat Stroke |
| Heart Rate | Cardiac Health, Metabolism, Athletic Performance, |
| | Weight Loss, Stress |

In some embodiments, the system 102 receives the input data from one or more user devices 104. The user devices 104 can be any device associated with a user (e.g., a patient or other operator), and can be used to obtain healthcare information, contextual information, and/or any other relevant information relating to the user and/or any other users (e.g., appropriately anonymized patient data). In the illustrated embodiment, for example, the user devices 104 include at least one biosensor 104a (e.g., blood glucose sensors, pressure sensors, heart rate sensors, sleep trackers, temperature sensors, motion sensors, or other biomonitoring devices), at least one mobile device 104b (e.g., a smartphone or tablet computer), and/or at least one wearable device 104c (e.g., a smartwatch, fitness tracker). In other embodiments, however, one or more of the devices 104a-c can be omitted and/or other types of user devices can be included, such as computing devices (e.g., personal computers, laptop computers, etc.). Moreover, although FIG. 1 illustrates the biosensor(s) 104a as being separate from the other user devices 104, in other embodiments the biosensor(s) 104a can be incorporated into another user device 104. Additional examples of biosensors 104a suitable for use with the present technology are described in greater detail below.

In some embodiments, some or all of the user devices 104 are configured to periodically or continuously obtain any of the above data (e.g., health-related information and/or contextual information) from the user over a particular time period (e.g., hours, days, weeks, months, years). For example, data can be obtained at a predetermined time interval (e.g., once every minute, 2 minutes, 5 minutes, 10 minutes, 15 minutes, 20 minutes, 30 minutes, 60 minutes, 2 hours, etc.), at random time intervals, or combinations thereof. The time interval for data collection can be set by the user, by another user (e.g., a physician), by the system 102, or by a user device 104 itself (e.g., as part of an automated data collection program). The user devices 104 can obtain the data automatically or semi-automatically (e.g., by automatically prompting the patient to provide such data at a particular time), or from manual input by the user (e.g., without prompts from the user device 104). The continuous data may be provided to the system 102 at predetermined time intervals (e.g., once every minute, 2 minutes, 5 minutes, 10 minutes, 15 minutes, 20 minutes, 30 minutes, 60 minutes, 2 hours, etc.), continuously, in real-time, upon receiving a query, manually, automatically (e.g., upon detection of new data), semi-automatically, etc. The time interval at which a user device 104 obtains data may or may not be the same as the time interval at which the user device 104 transmits the data to the system 102.

The user devices 104 can obtain any of the above data and can provide output in various ways, such as using one or more of the following components: a microphone (either a separate microphone or a microphone imbedded in the device), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, a sensor (e.g., a sensor included in or operably coupled to the user device 104), and/or any other device. The data obtained by the user devices 104 can include metadata, structured content data, unstructured content data, embedded data, nested data, hard disk data, memory card data, cellular telephone memory data, smartphone memory data, main memory images and/or data, forensic containers, zip files, files, memory images, and/or any other data/information. The data can be in various formats, such as text, numerical, alpha-numerical, hierarchically arranged data, table data, email messages, text files, video, audio, graphics, etc. Optionally, any of the above data can be filtered, smoothed, augmented, annotated, or otherwise processed (e.g., by the user devices 104 and/or by the system 102) before being used.

In some embodiments, any of the above data can be queried by one or more of the user devices 104 from one or more databases (e.g., the database 106, a third-party database, etc.). The user devices 104 can generate a query and transmit the query to the system 102, which can determine which database may contain requisite information and then connect with that database to execute a query and retrieve appropriate information. In other embodiments, the user device 104 can receive data directly from the third-party database and transmit the received data to the system 102, or can instruct the third-party database to transmit the data to the system 102. In some embodiments, the system 102 can include various application programming interfaces (APIs) and/or communication interfaces that can allow interfacing between user devices 104, databases, and/or any other components.

Optionally, the system 102 can also obtain any of the above data from various third-party sources, for example, with or without a query initiated by a user device 104. In some embodiments, the system 102 can be communicatively coupled to various public and/or private databases that can store various information, such as census information, health statistics (e.g., appropriately anonymized), demographic information, population information, and/or any other information. Additionally, the system 102 can execute a query or other command to obtain data from the user devices 104 and/or access data stored in the database 106. The data can include data related to the particular user and/or a plurality of other users (e.g., health-related information, contextual information, etc.) as described herein.

The database 106 can be used to store various types of data obtained and/or used by the system 102. For example, any of the above data can be stored in the database 106. The database 106 can also be used to store data generated by the system 102, such as previous predictions or forecasts produced by the system 102. In some embodiments, the database 106 includes data for multiple users, such as at least 50, 100, 200, 500, 1000, 2000, 3000, 4000, 5000, or 10,000 different users. The data can be appropriately anonymized to ensure compliance with various privacy standards. The database 106 can store information in various formats, such as table format, column-row format, key-value format, etc. (e.g., each key can be indicative of various attributes associated with the user and each corresponding value can be indicative of the attribute's value (e.g., measurement, time, etc.)). In some embodiments, the database 106 can store a plurality of tables that can be accessed through queries generated by the system 102 and/or the user devices 104. The tables can store different types of information (e.g., one table can store blood glucose measurement data, another table can store user health data, etc.), where one table can be updated as a result of an update to another table.

For example, Table 2 below illustrates example health and/or behavioral data that may be provided to the system 102 and/or stored in the database 106. The data in Table 2 can be generated by one or more user devices 104, as previously described. Each entry in Table 2 is labeled with a user ID, and includes a time stamp indicating when the data was obtained, the type of data, and the data value.

TABLE 2
Health and Behavioral Data

| User ID | Time | Data Type | Value | |
|---|---|---|---|---|
| user1 | 2018 08 30 7:48:15.124 utc | blood glucose | 135 | mg/dL |
| user2 | 2018 08 30 7:48:15.126 utc | carbohydrates | 38 | g |
| user3 | 2018 08 30 7:48:16.324 utc | activity | 30 | min |
| user2 | 2018 08 30 7:48:17.128 utc | medicine: insulin | 6 | U |
| user4 | 2018 08 30 7:48:15.226 utc | blood glucose | 218 | mg/dL |
| user1 | 2018 08 30 7:48:15.829 utc | carbohydrates | 14 | g |

| | | | |
|---|---|---|---|
| user5 | 2018 08 30 7:48:17.155 utc | a1c | 7.80% |

As another example, Table 3 below illustrates example personal data that may be provided to the system 102 and/or stored in the database 106. The data in Table 3 can be generated by one or more user devices 104, as previously described. Each entry in Table 3 is labeled with a user ID, and includes personal information for that particular user such as the time zone in which the user is located, the type of diabetes the user has, the date that the user was first enrolled in the system 102, the year in which the user was diagnosed with diabetes, and the user's gender.

TABLE 3
Personal Data

| | | Diabetes | | Diagnosis | |
|---|---|---|---|---|---|
| User ID | Time Zone | Type | Start Date | Year | Gender |
| user1 | New York | Type 2 | 2014 Mar. 5 | 2002 | F |
| user2 | Los Angeles | Type 1 | 2016 Dec. 26 | None | M |
| user3 | Mumbai | Type 2 | 2015 Apr. 8 | 2015 | None |
| user4 | Lisbon | Type 2 | 2017 Sep. 13 | None | M |

In some embodiments, one or more users can access the system 102 via the user devices 104, for example, to send data to the system 102 (e.g., health-related information, contextual information) and/or receive data from the system 102 (e.g., predictions, notifications, recommendations, instructions, support, etc.). The users can be individual users (e.g., patients, healthcare professionals, etc.), computing devices, software applications, objects, functions, any other types of users, and/or any combination thereof. For example, upon obtaining any of the input data discussed above, a user device 104 can generate an instruction and/or command to the system 102, for example, to process the obtained data, store the data in the database 106, extract additional data from one or more databases, and/or perform analysis of the data. The instruction/command can be in a form of a query, a function call, and/or any other type of instruction/command. In some implementations, the instructions/commands can be provided using a microphone (either a separate microphone or a microphone imbedded in the user device 104), a speaker, a screen (e.g., using a touchscreen, a stylus pen, and/or another suitable input instrument), a keyboard, a mouse, a camera, a camcorder, a telephone, a smartphone, a tablet computer, a personal computer, a laptop computer, and/or any other suitable device. The user device 104 can also instruct the system 102 to perform an analysis of data stored in the database 106 and/or inputted via the user device 104.

As discussed further below, the system 102 can analyze the obtained input data, including historical data, current real-time data, continuously supplied data, and/or any other data (e.g., using a statistical analysis, machine learning analysis, etc.), and generate output data. The output data can include predictions of a user's health state, correlations between data, interpretations, recommendations, notifications, instructions, support, and/or other information related to the obtained input data. In some embodiments, the output data provides information to assist the user in adjusting their behavior (e.g., diet, exercise, sleeping, etc.) to enhance outcomes; to reduce, limit, or avoid healthcare provider intervention; etc.

The system 102 can perform such analyses at any suitable frequency and/or any suitable number of times (e.g., once, multiple times, on a continuous basis, etc.). For example, when updated input data is supplied to the system 102 (e.g., from the user devices 104), the system 102 can reassess and update its previous output data, if appropriate. In performing its analysis, the system 102 can also generate additional queries to obtain further information (e.g., from the user devices 104, the database 106, or third-party sources). In some embodiments, a user device 104 can automatically supply the system 102 with such information. Receipt of updated and/or additional information can automatically trigger the system 102 to execute a process for reanalyzing, reassessing, or otherwise updating previous output data.

In some embodiments, the system 102 is configured to analyze the input data and generate the output data using one or more machine learning models. The machine learning models can include supervised learning models, unsupervised learning models, semi-supervised learning models, and/or reinforcement learning models. Examples of machine learning models suitable for use with the present technology include, but are not limited to: regression algorithms (e.g., ordinary least squares regression, linear regression, logistic regression, stepwise regression, multivariate adaptive regression splines, locally estimated scatterplot smoothing), instance-based algorithms (e.g., k-nearest neighbor, learning vector quantization, self-organizing map, locally weighted learning support vector machines), regularization algorithms (e.g., ridge regression, least absolute shrinkage and selection operator, elastic net, least-angle regression), decision tree algorithms (e.g., classification and regression trees, Iterative Dichotomiser 3 (ID3), C4.5, C5.0, chi-squared automatic interaction detection, decision stump, M5, conditional decision trees), Bayesian algorithms (e.g., naïve Bayes, Gaussian naïve Bayes, multinomial naïve Bayes, averaged one-dependence estimators, Bayesian belief networks, Bayesian networks), clustering algorithms (e.g., k-means, k-medians, expectation maximization, hierarchical clustering), association rule learning algorithms (e.g., apriori algorithm, ECLAT algorithm), artificial neural networks (e.g., perceptron, multilayer perceptrons, back-propagation, stochastic gradient descent, Hopfield networks, radial basis function networks), deep learning algorithms (e.g., convolutional neural networks, recurrent neural networks, long short-term memory networks, stacked auto-encoders, deep Boltzmann machines, deep belief networks), dimensionality reduction algorithms (e.g., principle component analysis, principle component regression, partial least squares regression, Sammon mapping, multidimensional scaling, projection pursuit, discriminant analysis), time series forecasting algorithms (e.g., exponential smoothing autoregressive models, autoregressive with exogenous input (ARX) models, autoregressive moving average (ARMA) models, autoregressive moving average with exogenous inputs (ARMAX) models, autoregressive integrated moving average (ARIMA) models, autoregressive conditional heteroscedasticity (ARCH) models), and ensemble algorithms (e.g., boosting, bootstrapped aggregation, AdaBoost, blending, stacking, gradient boosting machines, gradient boosted trees, random forest).

Although FIG. 1 illustrates a single set of user devices 104, it will be appreciated that the system 102 can be operably and communicably coupled to multiple sets of user devices 104, with each set being associated with a particular patient or user. Accordingly, the system 102 can be configured to receive and analyze data from a large number of users (e.g., at least 50, 100, 200, 500, 1000, 2000, 3000, 4000, 5000, or 10,000 different users) over an extended time period (e.g., weeks, months, years). The data from these patients can be used to train and/or refine one or more machine learning models implemented by the system 102, as described below.

The system 102 and the user devices 104 can be operably and communicatively coupled to each other via the network 108. The network 108 can be or include one or more communications networks, and can include at least one of the following: a wired network, a wireless network, a metropolitan area network (“MAN”), a local area network (“LAN”), a wide area network (“WAN”), a virtual local area network (“VLAN”), an internet, an extranet, an intranet, any other type of network, and/or any combination thereof. Additionally, although FIG. 1 illustrates the system 102 as being directly connected to the database 106 without the network 108, the system 102 can be indirectly connected to the database 106 via the network 108 in other embodiments. In these and other embodiments, one or more of the user devices 104 can be configured to communicate directly with the system 102 and/or the database 106 (e.g., in addition to or in lieu of communicating with these components via the network 108).

The various components 102-108 illustrated in FIG. 1 can include any suitable combination of hardware and/or software. In some embodiments, components 102-108 can be disposed on one or more computing devices, such as server(s), database(s), personal computer(s), laptop(s), cellular telephone(s), smartphone(s), tablet computer(s), any other suitable computing devices, and/or any combination thereof. In some embodiments, the components 102-108 can be disposed on a single computing device and/or can be part of a single communications network. Alternatively, the components can be located on distinct and separate computing devices. For example, although FIG. 1 illustrates the system 102 as being a single component, in other embodiments the system 102 can be implemented across a plurality of different hardware components at different locations.

### Biosensors and Associated Systems, Devices, and Methods

The systems and methods of the present technology can use one or more biosensors (also referred to herein as “biosensor devices,” “sensors,” or “sensor devices”) to generate user data, such as data indicative of a user's health state. The biosensors described herein can be or include various types of sensors, such as chemical sensors, electrochemical sensors, optical sensors (e.g., optical enzymatic sensors, opto-chemical sensors, fluorescence-based sensors, etc.), spectrophotometric sensors, spectroscopic sensors, polarimetric sensors, calorimetric sensors, iontophoretic sensors, radiometric sensors, and the like, and combinations thereof. The biosensors can be or include implanted sensors, non-implanted sensors, invasive sensors, minimally invasive sensors, non-invasive sensors, wearable sensors, etc. Additionally, or alternatively, the biosensors can be or include disposable sensors, reusable sensors, or any suitable combination of disposable and reusable components (e.g., a disposable sensor portion for monitoring specific conditions and a reusable or disposable electronics portion for receiving and processing the sensor data).

The number, configuration, and/or functionality of the biosensors can be selected based on desired sensing capabilities. For example, the biosensors described herein can be configured to sense any suitable combinations of the following health parameters: glucose, oxygen (e.g., oxygen, carbon dioxide, etc.), electrolytes (e.g., bicarbonate, potassium, sodium, magnesium, chloride, lactic acid), iodide, iodine, BUN, creatinine, ketones, cholesterol, triglycerides, alcohols, ethanol, amino acids, neurotransmitters, hormones, disease biomarkers (e.g., cancer biomarkers, cardiovascular disease biomarkers), drugs (e.g., concentrations, metabolism), pH, cell count, blood chemistry (e.g., analyte concentrations), vitals (e.g., heart rate, body temperature (such as skin temperature), blood pressure (such as systolic and/or diastolic blood pressure), respiratory rate, blood saturation levels (such as blood oxygen saturation)), cardiovascular data (e.g., pacemaker data, arrhythmia data), body function data, meal or nutrition data (e.g., number of meals; timing of meals; number of calories; amount of carbohydrates, fats, sugars, etc.), physical activity or exercise data (e.g., time and/or duration of activity; activity type such as walking, running, swimming; strenuousness of the activity such as low, moderate, high; etc.), sleep data (e.g., number of hours of sleep, average hours of sleep, variability of hours of sleep, sleep-wake cycle data, data related to sleep apnea events, sleep fragmentation (such as fraction of nighttime hours awake between sleep episodes, etc.)), stress level data (e.g., cortisol and/or other chemical indicators of stress levels, perspiration), al c data, user location (e.g., GPS coordinates, elevation data), environmental conditions (e.g., air pressure, humidity, temperature, air quality, etc.), or combinations thereof.

In some embodiments, the biosensor can be or include a blood glucose sensor. The blood glucose sensor can be any device capable of obtaining blood glucose data from a user. The blood glucose sensor can be configured to obtain samples from the user (e.g., blood samples, interstitial fluid samples) and determine glucose levels in the sample. Any suitable technique for obtaining user samples and/or determining glucose levels in the samples can be used. In some embodiments, the blood glucose sensor can be configured to detect substances (e.g., a substance indicative of glucose levels), measure a concentration of glucose, and/or measure another substance indicative of the concentration of glucose. The blood glucose sensor can be configured to analyze, for example, body fluids (e.g., blood, interstitial fluid, sweat, etc.), tissue (e.g., optical characteristics of body structures, anatomical features, skin, or body fluids), and/or vitals (e.g., heat rate, blood pressure, etc.) to periodically or continuously obtain blood glucose data. Optionally, the blood glucose sensor can include other capabilities, such as processing, transmitting, receiving, and/or other computing capabilities. In some embodiments, the blood glucose sensor can include at least one continuous glucose monitoring (CGM) device or sensor that measures the user's blood glucose level at predetermined time intervals. For example, the CGM device can obtain at least one blood glucose measurement every minute, 2 minutes, 5 minutes, 10 minutes, 15 minutes, 20 minutes, 30 minutes, 60 minutes, 2 hours, etc. In some embodiments, the time interval is within a range from 5 minutes to 10 minutes.

The biosensors described herein can include various functionalities to facilitate data collection and/or processing. For example, the biosensors can be configured to perform one or more of the following functions: compensate for biofouling associated with body fluid-based monitoring deliver medication, reduce or limit signal noise, compensate for time delays (e.g., with glucose changes for signal detection associated with body fluid-based detection), and/or manage over the air updates (e.g., algorithm updates, detection updates, software module updates). Biosensors of the present technology can include electronics for detecting sensors and/or for detecting different analytes, and/or can use additional information (e.g., exercise, food, etc.) in algorithms. Detection can be performed using different algorithms used with different groups of users and/or algorithms selected based on user health data.

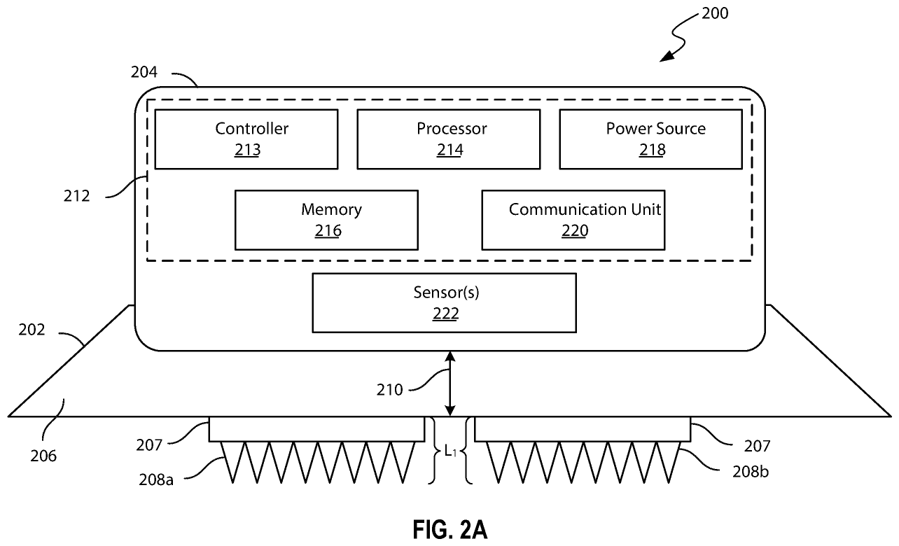

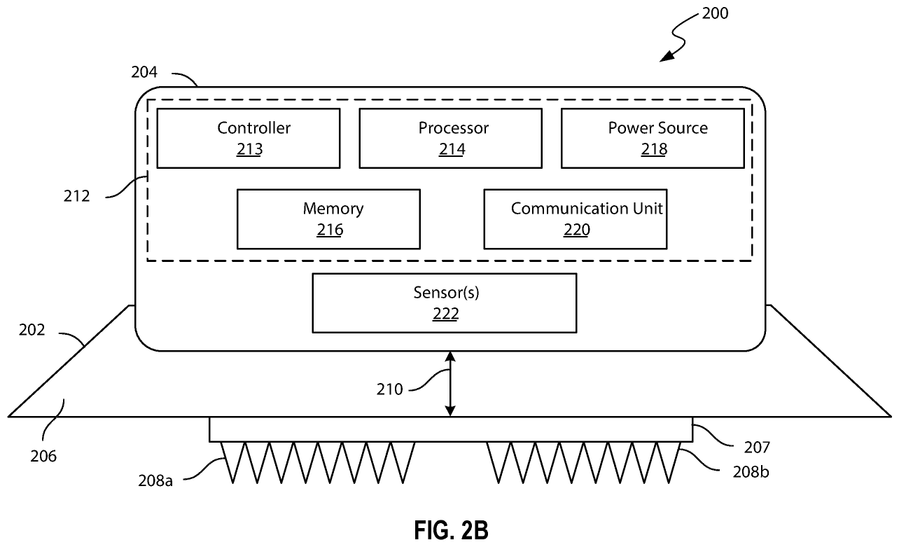

FIGS. 2A and 2B and the accompanying description provide various examples of biosensors that are suitable for use with the biomonitoring and healthcare guidance system 102 of FIG. 1. Specifically, FIGS. 2A and 2B are schematic illustrations of a biosensor device 200 (“device 200”) configured in accordance with various embodiments of the present technology. The device 200 can be a wearable patch sensor configured to be applied to a user's body in order to obtain user health data in a non-invasive or minimally invasive manner. The device 200 can be used in any of the systems and methods described herein (e.g., as the biosensor 104a of FIG. 1). The device 200 includes a patch 202 (also referred to as a “disposable patch,” “microsensor patch,” “microsensor,” “patch portion,” “base portion,” or “sensing component”) and a pod 204 (also referred to as a “reusable pod,” “pod portion,” “capsule portion,” or “electronics component”). The patch 202 can be coupled to the pod 204 (e.g., releasably coupled or permanently affixed) to form the device 200.

The patch 202 can include a substrate 206 configured to couple to the user's body (e.g., to the surface of the skin) via adhesives or other suitable temporary attachment techniques. The base portion also includes at least one array of microneedles 208 (two arrays are shown in FIGS. 2A and 2B, referred to individually as a first array of microneedles 208a and a second array of microneedles 208b) coupled to and/or supported by the substrate 206. The microneedles 208 can generally have a length L₁ (FIG. 2A) and can be configured to penetrate into the user's skin to access interstitial fluid therein. In some embodiments, when the device 200 is applied to the skin, the length L₁ is selected such that the microneedles 208 extend only into the stratum corneum and epidermis, and do not penetrate into the dermis or hypodermis (subcutaneous tissue). This approach can reduce or avoid pain and/or discomfort, while still providing accurate detection of analytes in the epidermal interstitial fluid. The microneedles 208 can be configured to detect one or more analytes in the interstitial fluid, such as glucose, gases, electrolytes, BUN, creatinine, ketones, alcohols, amino acids, neurotransmitters, hormones, biomarkers, drugs, pH, cell count, and/or any of the other analytes described herein. Each of the microneedles 208 can be configured to detect a single analyte, or some or all of the microneedles 208 can be configured to detect multiple analytes (e.g., two, three, four, five, or more different analytes). Optionally, some or all of the microneedles 208 can be configured to detect physiological parameters, such as electrical properties (e.g., biopotential, bioimpedance), body temperature, etc. In some embodiments the first array of microneedles 208a is configured to detect a first set of one or more analytes while the second array of microneedles 208b is configured to detect a second set of one or more analytes.

The array can include any suitable number of microneedles 208 (e.g., 25 microneedles), and the microneedles 208 can be arranged in any suitable geometry (e.g., a 5×5 grid) and/or the device 200 can include two, three, four, five, or more arrays of microneedles 208. Spacings between the microneedles 208 of an array can be uniform or can vary across the array and/or across multiple arrays. In embodiments where the device 200 includes multiple arrays, each array can be configured to perform a different function, or some of the arrays can perform the same function. For example, as discussed above, the first array of microneedles 208a can be configured to detect a first set of analytes, while the second array of microneedles 208b can be configured to detect a second set of analytes. Further, the device 200 can include a third array of microneedles 208 configured to detect a third set of analytes, and so on. Alternatively, or additionally, the first array of microneedles 208a can be configured as a working electrode, the second array of microneedles 208b can be configured as a reference electrode, and a third array of microneedles (not shown) can be configured as a counter electrode.

Referring to FIG. 2A, the first and second arrays of microneedles 208a, 208b are coupled to the substrate 206 through a base substrate 207 separated into two sections. For example, the separate configuration can be used when the first and second arrays of microneedles 208a, 208b are configured as separate electrodes (e.g., a working electrode and a reference electrode). In some embodiments, the first and second arrays of microneedles 208a, 208b are coupled to the sub state 206 through a single base substrate 207, as illustrated in FIG. 2B. For example, the single configuration can be used when the first and second arrays of microneedles 208a, 208b are configured to detect and/or respond to separate sets of analytes of interest and/or to simplify the construction of the device. The substrate 207 can include, without limitation, one or more layers, circuitry, pads, mounting features, etc. In some embodiments, the first and second arrays of microneedles 208a, 208b are integrally formed (e.g., via etching, cutting, build-up, etc.) with the base substrate 207.

Referring to FIGS. 2A and 2B together, the first and second arrays of microneedles 208a, 208b can each generate signals (e.g., electrical signals) indicative of health parameter values (e.g., analyte concentration and/or physiological values). For example, the first array of microneedles 208 can generate a first electrical signal indicative of a first analyte, a second electrical signal indicative of a second analyte, and so on. Optionally, either (or each) of the first and second arrays of microneedles 208a, 208b can generate at least a first electrical signal indicative of an analyte and at least a second electrical signal indicative of a physiological parameter. The first and second arrays of microneedles 208a, 208b can each be electrically coupled to the patch 202, which in turn can be electrically coupled to the pod 204 (schematically represented by arrow 210). The electrical connections between the first and second arrays of microneedles 208a, 208b, patch 202, and pod 204 can include any suitable combination of pins, contacts, wires, traces, etc. Accordingly, the signals generated by the microneedles 208 can be transmitted to the pod 204 for storage and/or processing.

The pod 204 can be a capsule, module, or other durable structure that couples to the patch 202 in order to assemble the device 200. The pod 204 can be mechanically coupled to the patch 202 using any suitable temporary or permanent attachment method, such as interference fit, snap fit, threading, fasteners, bonding, adhesives, and/or suitable combinations thereof. The pod 204 can include a casing or housing that encloses an electronics assembly 212 (also referred to herein as an “electronics subsystem”) of the device 200. The electronics assembly 212 can include one or more electronic components configured to perform the various operations described herein, such as a controller 213, processor 214, memory 216, power source 218, and communication unit 220. The controller 213 can be include any number of processors 214, memory 216, and other electronic components disclosed herein. Optionally, the pod 204 can also include one or more sensors 222 for measuring physiological parameters. The pod 204 can also include other electronic components not shown in FIGS. 2A and 2B, such as additional signal processing circuitry (e.g., multiplexer, analog front end (AFE), amplifier, filter, analog-to-digital converters (ADCs)), clock circuitry, power management circuitry, user input/output devices, and the like.

The processor 214 can be any component suitable for controlling the operations of the device 200, such as a microprocessor, microcontroller, field-programmable gate array (FPGA), application-specific integrated circuit (ASIC), and the like. For example, the processor 214 can receive and process signals generated by either (or both) of the first and second arrays of microneedles 208a, 208b and/or the sensor(s) 222 in order to generate one or more measurements of health parameters (e.g., analyte levels, biopotential values, bioimpedance values, body temperature values, heart rate values, oxygen levels, etc.). In some embodiments, the processor 214 receives and processes at least a first electrical signal from any of the microneedles 208 to generate a first health measurement (e.g., an analyte level), and at least a second electrical signal from the sensor(s) 222 to generate a second health measurement (e.g., a physiological parameter). The processor 214 can be configured to receive and process any number of electrical signals (e.g., two, three, four, five, or more electrical signals) obtained by different sensing components of the device 200 to generate measurements of multiple health parameters (e.g., two, three, four, five or more different health parameters). Optionally, the processor 214 can use the health measurements to generate predictions, recommendations, notifications, etc. As another example, the processor 214 can control transmission of raw sensor data, processed data, health measurements, predictions, etc., to a remote device (e.g., a smartphone, smartwatch, or other user device or remote server). In a further example, the processor 214 can receive instructions from a remote device for controlling the operation of the device 200 (e.g., powering on, powering off, updating calibration and/or other signal processing parameters, device pairing, etc.). The processor 214 can also control the operations of the other components of the device 200 (e.g., operations of the memory 216, power source 218, communication unit 220, other sensor(s) 222, etc.).

The memory 216 can store instructions to be executed by the processor 214 and/or data generated during operation of the device 200. For example, the memory 216 can store raw and/or processed sensor data, as well as generated health measurements, predictions, recommendations, notifications, etc. The memory 216 can also store operating parameters for the device 200, such as calibration parameters, signal processing parameters, algorithms or programs (e.g., for generating health measurements, predictions, etc.), and so on. The memory 216 can also store one or more unique identifiers associated with any of the components of the device 200. The memory 216 can include any suitable combination of volatile and non-volatile memory, such as flash memory, EEPROM, etc. The memory can store instructions that are executable by the processor 214 to, for example, analyze collected data, control operation of the microneedles 208 or the like to generate health measurements, predictions, recommendations, notifications. In some embodiments, the memory 216 stores disabling routines for disabling usage of the pod 204 and/or microneedles 208 in response to identifying a disabling event. The disabling event can include, but is not limited to, expired microneedles, needles, and the like; reused microneedles, needles, and the like; detected malfunctioning; improper placement of the device 200 on the user; operation errors; incorrect microneedles, needles, and the like (e.g., microneedles not configured to detect correct analytes); etc. For example, the pod 204 can receive a manufacture date, expiration date, and/or other suitable data to determine whether the microneedles 208 are past an expected shelf life. A reused patch 202 can be detected when installed and a notification can be sent to the user to help prevent the device 200 from being placed on the user. Malfunctioning can be detected before, during, or after placement on the user. Improper device placement can be detected upon installation or continued use. Placement and needle positions can be continuously or intermittently determined for short-term use, long-term use (e.g., over 4 weeks), etc. Routines can be configured based on the expected period of use and include, without limitation, calibration routines that compensate for one or more of production data, physiological changes, chemistry changes, power source levels, user settings, combinations thereof, or the like. The biosensor device 200 can include computer-readable media having computer-readable storage media (e.g., “non-transitory” media) and computer-readable transmission media.

The power source 218 can be any component suitable for powering the operations of the device 200, such as a rechargeable battery, non-rechargeable battery, or suitable combinations thereof. The power source 218 can output power to the first and second arrays of microneedles 208a, 208b, processor 214, memory 216, communication unit 220, sensor(s) 222, and/or any other electronic components on the patch 202 or pod 204. The power source 218 can include or be operably coupled to power management circuitry (not shown). The power management circuitry can detect the charge status of the power source 218 (e.g., fully charged, partially charged, low charge), can allow the device 200 to operate in various modes (e.g., low power, full power), and/or any other suitable power-related function.

The communication unit 220 can allow the device 200 to transmit data to and/or receive data from a remote device (e.g., a mobile device, smartwatch, remote server, etc.). The communication unit 220 can be configured to communicate via any suitable combination of wired and/or wireless communication modes. In some embodiments, for example, the communication unit 220 uses Bluetooth Low Energy (BLE) to transmit and receive data.

The sensor(s) 222 can include any suitable combination of sensors for monitoring various health parameters, such as an optical sensor (e.g., photoplethysmography (PPG) sensor, pulse oximeter), heart rate sensor, blood pressure sensor, electrocardiogram (ECG) sensor, activity or motion sensor (e.g., accelerometer, gyroscope), temperature sensor (e.g., thermistor), location sensor, humidity sensor, etc. Each sensor can generate a respective set of signals, which can be received and processed by the processor 214 to generate health measurements and/or other user data. In some embodiments, the device 200 includes at least one, two, three, four, five, or more different sensors 222 for measuring physiological and/or other user parameters. Each sensor 222 can be located at any suitable region of the pod 204, such as at or near an upper surface, lower surface, lateral surface, or within an interior cavity of the pod 204. In other embodiments, however, some or all of the sensor(s) 222 can instead be located in the patch 202, rather than in the pod 204. For example, a temperature sensor can be located in the patch 202 in order to generate measurements of the user's skin temperature.

In some embodiments, the patch 202 is a disposable component that is configured for short-term use (e.g., no more than 4 weeks, 3 weeks, 2 weeks, 1 week, 6 days, 5 days, 4 days, 3 days, 2 days, 1 day, 12 hours, etc.), while the pod 204 is a reusable component that is configured for longer-term use (e.g., at least 1 week, 2 weeks, 3 weeks, 4 weeks, 1 month, 2 months, 3 months, 6 months, 1 year, etc.). This approach can be advantageous for reducing overall cost of the device 200, particularly in embodiments where the pod 204 includes more expensive components (e.g., the electronics assembly 212 and/or other sensor(s) 222). In such embodiments, the reusable pod 204 can be coupled to the disposable patch 202 to assemble the device 200 for use, and can be decoupled from the disposable patch 202 when the disposable patch 202 is to be replaced. As such, a single reusable pod 204 can be used with multiple different disposable patches 202, which can reduce the overall cost of the device 200, and enhance device longevity and adaptability. Optionally, a single reusable pod 204 can be used with multiple disposable patches 202 that detect different types of analytes. For example, the reusable pod 204 can be configured to interface with a first disposable patch 202 configured to detect a first set of analytes, a second disposable patch 202 configured to detect a second set of analytes, a third disposable patch 202 configured to detect a third set of analytes, and so on. In other embodiments, however, the patch 202 and pod 204 can both be disposable components, or can both be reusable components.

The device 200 can be configured to obtain and process the signals generated by the first and second arrays of microneedles 208a, 208b and/or the sensor(s) 222 in order to determine measurements for one or more health parameters, such as measurements of glucose, gases, electrolytes, BUN, creatinine, ketones, cholesterol, alcohols, amino acids, neurotransmitters, hormones, disease biomarkers, drugs, pH, cell count, heart rate, body temperature, blood pressure, respiratory rate, cardiovascular data, body function data, meal or nutrition data, physical activity or exercise data, sleep data, stress level data, al c data, and so on. In some embodiments, the electronics assembly 212 is configured to implement one or more algorithms, such as algorithms for sensor calibration, signal conditioning, determining presence of and/or values for health parameters based on the sensor signals, predicting current and/or future values for health parameters based on the sensor signals, etc. The algorithms can be stored locally at the electronics assembly 212 (e.g., in the memory 216) such that the device 200 can operate without being in communication with a separate computing device or system (e.g., a cloud computing network, remote server, user device, etc.). In such embodiments, the locally stored algorithms can be periodically updated, e.g., via firmware updates and/or other modifications received from the separate computing device by the communication unit 220. Alternatively, or in combination, some or all of the algorithms can be stored at the separate computing device or system. In some embodiments, local processing (e.g., using artificial-intelligence and/or machine-learning trained algorithms, or other suitable algorithms) can be performed onboard the device 200 for certain situations (e.g., when network connectivity is lost), while processing can be performed at a separate computing device or system in other situations (e.g., when network connectivity is available).

The operation of the device 200 can be customized based on the particular health parameters to be detected. For example, the patch 202 can include a respective memory (not shown) configured to store identifier information for the patch 202, such as the type and/or configuration of the microneedles 208, the type and/or configuration of the microneedle arrays, the types of analytes and/or physiological parameters detected by the microneedles 208, the types of other sensors included in the patch 202, a unique patch ID (e.g., a serial number), a lot ID, manufacturing date, expiration date and/or expected lifetime, and/or any other suitable information. In some embodiments, the processor 214 is configured to detect when the pod 204 is coupled to the patch 202. Once the pod 204 is connected to the patch 202, the processor 214 can interrogate or otherwise communicate with the patch 202 to detect the identifier information for the patch 202. The processor 214 can access and read the identifier information, and can then adjust the parameters and/or algorithms used to process the electrical signals generated by the patch 202 (e.g., by the microneedles 208), based on the identifier information. For example, the processor 214 can use the identifier information to determine detection capabilities of the patch 202 (e.g., which analytes and/or physiological values the patch 202 is configured to detect). The processor 214 can select an appropriate locally stored algorithm for processing the signals generated by the patch 202 and/or determining health parameters from the signals. The algorithm can vary depending on the microneedle type and/or configuration, type of detected analyte or parameter, the manufacturing information for the patch 202 (e.g., batch or lot ID), the expected lifetime of the patch 202, other available sensor data, or any other suitable factor. Additionally, parameter detection can be performed using different algorithms used with different groups of users and algorithms selected based on user health data. The locally stored algorithms can be updated based on the health parameters (e.g., via updates received from a separate user device, cloud computing system, etc.).

In embodiments where the pod 204 is configured for use with multiple patches 202 having different functionalities (e.g., different detection capabilities), the processor 214 can, when the pod 204 is coupled to a new patch 202, use the identifier information received from the patch 202 to assess the functionality of the patch 202. If the processor 214 determines that the patch 202 has newly available functionality that the processor 214 is not currently programmed to accommodate, the processor 214 can retrieve the appropriate algorithms, calibration parameters, signal processing parameters, and/or other updates from a remote device (e.g., a user device, cloud computing system, etc.). Accordingly, the software implemented by the pod 204 can be rapidly and dynamically updated to accommodate different and/or new patch functionalities.

The health measurements produced by the device 200 can be used to generate personalized healthcare guidance, such as one or more predictions, recommendations, suggestions, feedback, and/or diagnosis fora number of diseases, conditions, or health states. For example, blood pressure can be monitored and/or predicted based on optical data (e.g., PPG data), electrical data (e.g., ECG data), heart rate data, user data, and/or activity data. As another example, sleep (e.g., sleep patterns, sleep quality) can be tracked and/or predicted based on heart rate data, skin temperature data, and/or activity data. In a further example, respiratory illness (e.g., COVID-19, allergies, infections etc.) can be monitored and/or predicted based on skin temperature data, blood pressure data, and/or respiration rate. The health measurements can be used to detect a condition, distinguish between different conditions (e.g., infection versus allergies), and/or monitor the progression of the condition. In yet another example, fertility can be tracked and/or predicted based on skin temperature data. The personalized guidance can be generated based solely on the health measurements from the device 200, or can be generated through a combination of health measurements and other information (e.g., information from any number of sensor data streams, user data sets, etc.). The healthcare guidance can be generated locally onboard the device 200, by a user device that receives health measurement data from the device 200 (e.g., via a mobile application on a user's smartphone or smartwatch), by a cloud computing system or remote server that receives health measurement data from the device 200, or any suitable combination thereof.

The configuration of the device 200 shown in FIGS. 2A and 2B can be modified in many different ways. For example, in other embodiments, the array of microneedles 208 can be omitted such that the device 200 does not include or otherwise use microneedle-based analyte detection. In such embodiments, the patch 202 may include other sensor types (e.g., a temperature sensor, a metal electrode for ECG sensing), or may not include any sensors at all, such that all sensing operations are performed by the sensor(s) 222 (e.g., motion sensor, optical sensor, etc.) located in the pod 204.

In an alternative arrangement of the present technology, the patch 202 can include a one or more skin-penetrating needles (not shown) in addition to or in lieu of the microneedles 208. The skin-penetrating needle(s) can have a length L₂ that is significantly longer than the length L₁ of the microneedles 208 discussed above with reference to FIGS. 2A and 2B. Accordingly, the needle(s) can be configured to penetrate into and/or through the dermis or hypodermis (subcutaneous tissue) of the user to access a blood sample and/or interstitial fluid (e.g., subcutaneous interstitial fluid) within the user's body. While this configuration can be associated with increased installation pain for the user and some discomfort while deployed, this configuration can access fluids with higher concentrations of analytes of interest. As a result, the device 200 may be able to provide accurate measurements through the needle(s) more easily than through the microneedles 208 illustrated in FIGS. 2A and 2B.

A needle can be a single-analyte or multiple-analyte needle. For example, a needle can be a multi-analyte detecting needle that includes a plurality of detecting regions or electrodes (e.g., three regions or electrodes) that can each be electrically and/or chemically isolated from each other. As a result, similar to the discussion above, each of the electrodes can be configured differently. Purely by way of example, a first electrode can be configured as a first working electrode for detecting a first set of analytes (e.g., glucose, gases, electrolytes, BUN, creatinine, ketones, alcohols, amino acids, neurotransmitters, hormones, biomarkers, drugs, pH, cell count, and/or any combination therein), a second electrode can be configured as a reference electrode, and a third electrode can be configured as a counter electrode. In some embodiments, the needle can additionally or alternatively include various active regions, layers, and/or other components.

As another example of an alternative arrangement, any of the components of the device 200 discussed above with reference to FIGS. 2A and 2B can be separated into discrete subcomponents (e.g., multiple processors 214, multiple memories 216, etc.), combined into a single component (e.g., the processor 214 and communication unit 220 can be integrated into a single chip), or omitted altogether. In a still further example, any of the components of the device 200 can be positioned at different locations (e.g., some or all of the sensor(s) 222 can be located on the patch 202 instead of the pod 204). In some embodiments, the device 200 can include a combination of one or more arrays of the microneedles 208 and one or more needles. For example, the first and second arrays of microneedles 208a, 208b can detect analyte(s) in shallow tissue (e.g., dermis, epidermis, etc.) and the needle(s) can detect one or more analytes in deeper tissue. The number, detection capabilities, dimensions, and features of the needles can be selected based on target detection capabilities. Additionally, the microneedles 208 and/or the needles can be in the form of electrodes or sensing elements delivered into tissue using, for example, delivery needles, puncturing elements, etc.

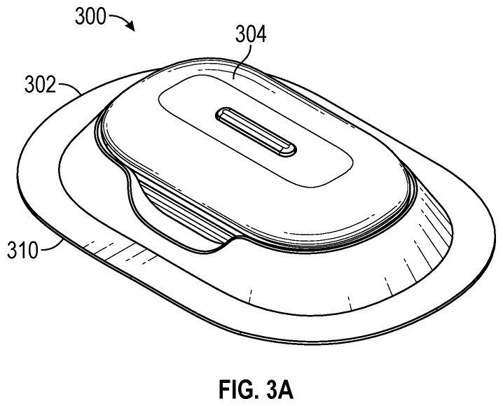

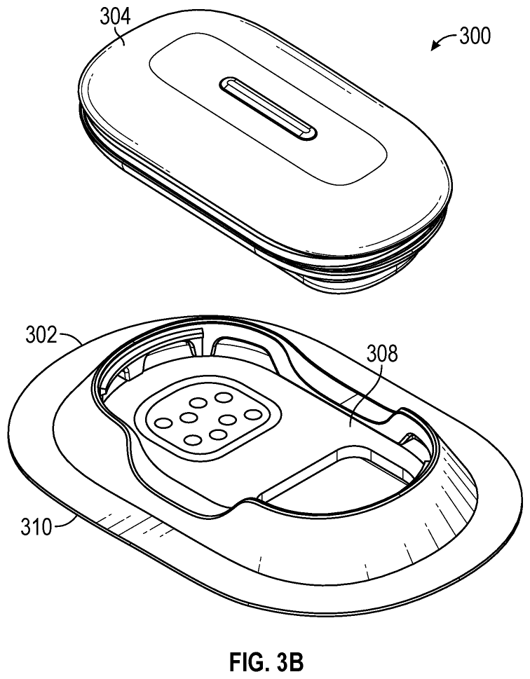

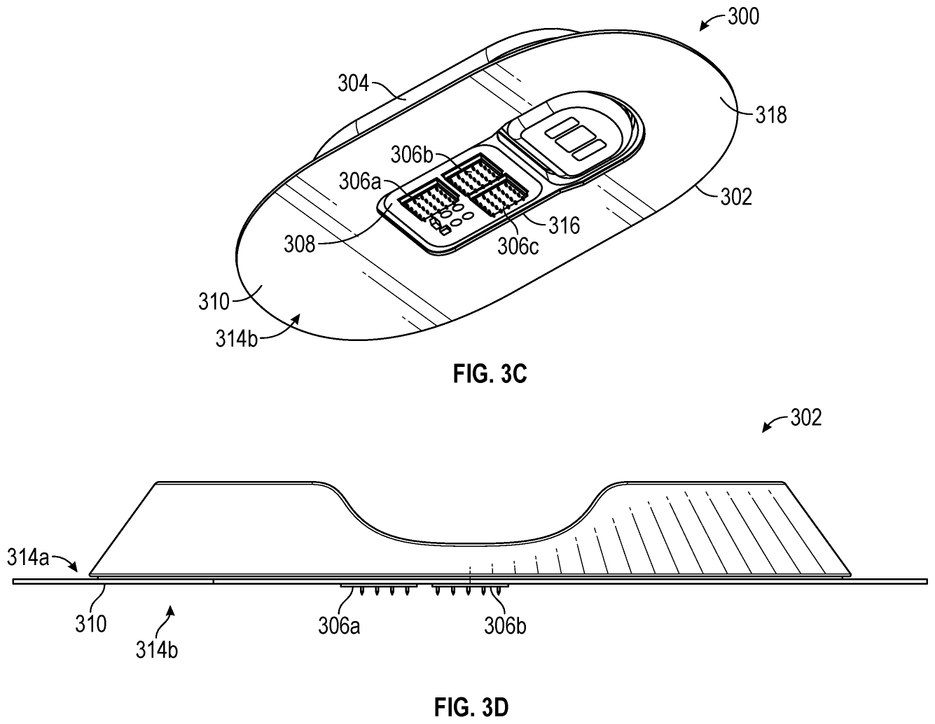

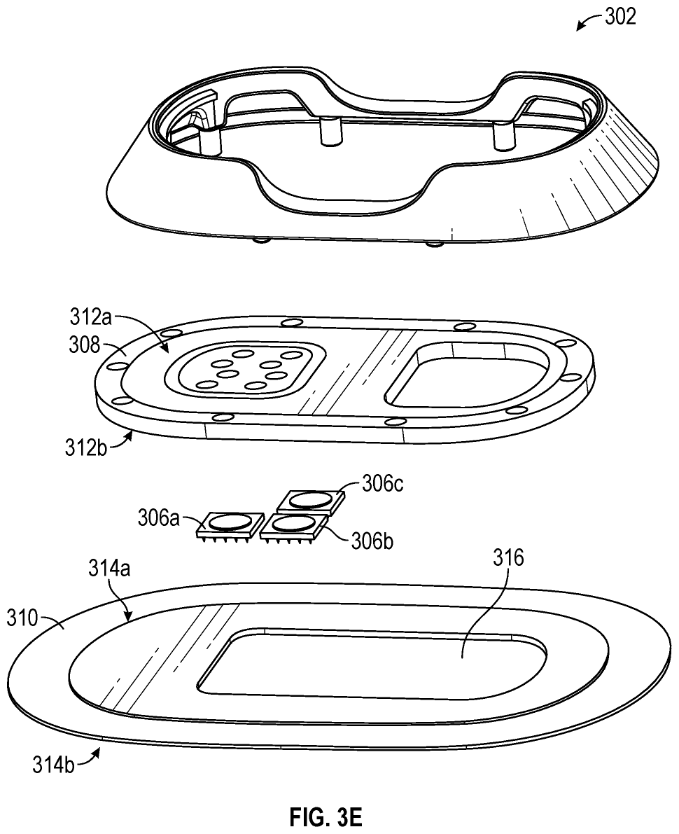

FIGS. 3A-3E illustrate a representative example of a biosensor device 300 (“device 300”) configured in accordance with various embodiments of the present technology. Specifically, FIGS. 3A-3C illustrate the overall device 300, and FIGS. 3D and 3E illustrate a patch portion 302 (“patch 302,” sometimes also referred to herein as a “microsensor patch” and/or a “disposable patch”) of the device 300. The description of the biosensor device 104a (FIG. 1) and devices 200 (FIGS. 2A and 2B) applies equally to the biosensor device 300.

Referring first to FIG. 3A (top perspective view), 3B (exploded view), and 3C (bottom perspective view) together, the device 300 is configured as a wearable sensor for application to a user's body. The device 300 includes a patch 302 for mounting to the skin, and a pod 304 that interfaces with the patch 302. The patch 302 and the pod 304 can be discrete components that are releasably connected to each other to form the device 300 (FIGS. 3A and 3C show the device 300 when assembled, and FIG. 3B shows the device 300 when the patch 302 and the pod 304 are separated). As previously discussed, the patch 302 can be a disposable component intended for short-term use, while the pod 304 can be a reusable component intended for longer-term use with multiple different patches 302.

The device 300 can be configured to be worn by the user over an extended period of time in order to generate measurements of any of the health parameters described herein, such as analyte levels (e.g., concentrations of glucose, gases, electrolytes, BUN, creatinine, ketones, cholesterol, triglycerides, alcohols, amino acids, neurotransmitters, hormones, disease biomarkers, drugs, etc.), physiological information (e.g., heart rate, body temperature, blood oxygenation, blood pressure, respiratory rate, bioimpedance, activity levels, sleep data), etc. In some embodiments, the device 300 includes a plurality of different sensor types for measuring multiple health parameters. For example, the device 300 can include at least two, three, four, five, or more different sensor types. The sensors can be located in the patch 302, the pod 304, or any suitable combination thereof.

FIG. 3D is a side view of the patch 302, and FIG. 3E is an exploded view of the patch 302. Referring to FIGS. 3B-3E together, the patch 302 is configured to temporarily attach to the user's body, such as on the skin of the user's hand, arm, shoulder, leg, foot, chest, back, neck, etc. The patch 302 can include one or more sensors that generate signals indicative of analyte levels, physiological parameters, and/or other health parameters associated with the user's skin. As best seen in FIG. 3C, the patch 302 can include sets of microneedles 306a-306c (e.g., arrays or microneedles) configured to penetrate into the user's skin (e.g., into the epidermis). The sets of microneedles 306a-c can incorporate any of the features described above with respect to the microneedles 208 of FIGS. 2A and 2B.

In the illustrated embodiment, the patch 302 includes three sets of microneedles 306a-c, each including 25 microneedles arranged in a 5×5 grid. The sets of microneedles 306a-c can be configured to detect one or more analytes in the interstitial fluid of the epidermis, for example, using electrochemical techniques. For example, the set 306a can be configured as a first working electrode for detecting a first set of analytes (e.g., glucose), the set 306b can be configured as a reference electrode, and the set 306c can be configured as a counter electrode. In other embodiments, however, the patch 302 can include fewer or more sets of microneedles, and/or the configuration (e.g., geometry, number of microneedles, position or spacing of microneedles, detected analyte, etc.) of each set can be varied as desired. For example, the patch 302 can include four sets of microneedles, with two sets configured as working electrodes, one set configured as a reference electrode, and one set configured as a counter electrode.

Optionally, some or all of the sets of microneedles 306a-c can alternatively or additionally detect other parameters besides analyte concentration, such as bioimpedance, biopotential, etc. For example, bioimpedance can be used to assess various physiological parameters, such as respiration rate, body composition, and/or hydration. Additionally, bioimpedance measurements of individual microneedles and/or sets of microneedles 306a-c can be used to measure or estimate microneedle penetration into the skin (e.g., whether the sets of microneedles 306a-c are in proper contact with the skin, the percentage of microneedles in each array that are in proper contact with the skin, etc.). The amount of microneedle penetration can be used to adjust downstream signal processing performed by the device 300, such as selecting correction factors for signal processing algorithms, selecting the algorithms to be used, selecting subsets of data to be used or excluded, etc.

As best shown in FIG. 3E, the patch 302 can include an electronics substrate 308 (e.g., a printed circuit board (PCB), a flex circuit, etc.) and a mounting substrate 310 (e.g., an adhesive film, sticker, tape, etc.) that collectively support the sets of microneedles 306a-c and couple to the user's body. The electronics substrate 308 can be a flattened, oval-shaped structure having an upper surface 312a and a lower surface 312b, and the mounting substrate 310 can also be a flattened, oval-shaped structure having an upper surface 314a and a lower surface 314b. In other embodiments, the electronics substrate 308 and mounting substrate 310 can each independently have a different shape (e.g., circular, square, rectangular, etc.). Additionally, although the mounting substrate 310 is illustrated as being larger than the electronics substrate 308 (e.g., with respect to length, width, perimeter, etc.), in other embodiments, the mounting substrate 310 can be the same size as the electronics substrate 308 or can be smaller than the electronics substrate 308. Moreover, in other embodiments, the electronics substrate 308 and mounting substrate 310 can be combined into a single, unitary component, rather than being two discrete components that are connected to each other to assemble the patch 302.

The sets of microneedles 306a-c can be coupled to the lower surface 312b of the electronics substrate 308. The mounting substrate 310 can include an aperture 316 configured such that, when the lower surface 312b of the electronics substrate 308 is attached to the upper surface 314a of the mounting substrate 310, the microneedles of the sets 306a-c pass through the aperture 316 and extend past the lower surface 314b of the mounting substrate 310 in order to access the user's skin (as best shown in FIGS. 3C and 3D). Optionally, the aperture 316 of the mounting substrate 310 can be larger than the surface area of the sets of microneedles 306a-c so that one or more additional sensors can extend through the mounting substrate 310 to access the skin.

Referring to FIG. 3C, the mounting substrate 310 can be configured to temporarily secure the patch 302 (as well as the rest of the device 300) to the user's skin. For example, the lower surface 314b of the mounting substrate 310 can include an adhesive region 318 configured to temporarily attach to the user's skin. The adhesive region 318 can extend across the entirety of the lower surface 314b, or at least portions thereof. In the illustrated embodiment, the sets of microneedles 306a-c are located at the central portion of the mounting substrate 310, such that the adhesive region 318 completely surrounds the sets of microneedles 306a-c to maintain the sets of microneedles 306a-c in close contact with the skin. In other embodiments, however, the sets of microneedles 306a-c and/or the adhesive region 318 can be arranged differently. For example, the sets of microneedles 306a-c can be offset to one side of the mounting substrate 310 and/or the adhesive region 318 can surround only a portion of the sets of microneedles 306a-c. The adhesive region 318 can be made of any suitable material suitable for coupling to the skin for an extended time period (e.g., at least 12 hours, 24 hours, 2 days, 3 days, 4 days, 5 days, 6 days, 1 week, etc.). The material of the adhesive region 318 can be biocompatible, breathable, and/or water-resistant, for example, to reduce discomfort and/or avoid premature detachment. In some embodiments, the mounting substrate 310 itself can be a flexible component configured to conform the user's body to further improve adhesion and user comfort. Additional details regarding the device 300 illustrated in FIGS. 3A-3E are disclosed in U.S. application Ser. No. 17/236,753 (U.S. Pub. No. 2021/0321942) and U.S. application Ser. No. 17/578,386, the entire disclosures of which are hereby incorporated by reference herein in their entireties.

Additional details on biosensors, methods of biomonitoring, and related technologies are disclosed in U.S. Pat. Nos. 9,008,745; 9,182,368; 10,173,042; 10,595,754; U.S. application Ser. No. 15/876,678 (U.S. Pub. No. 2018/0140235); U.S. application Ser. No. 14/812,288 (U.S. Pub. No. 2016/0029931); U.S. application Ser. No. 14/812,302 (U.S. Pub. No. 2016/0029966); U.S. Pat. No. 10,820,860; U.S. application Ser. No. 16/888,105 (U.S. Pub. No. 2020/0375549); U.S. application Ser. No. 16/558,558 (U.S. Pub. No. 2020/0077931); and U.S. application Ser. No. 17/167,795 (U.S. Pub. No. 2021/0241916), the disclosures of which are all hereby incorporated by reference in their entireties. These technologies can be used with, incorporated into, and/or combined with systems, methods, features, and components disclosed herein. Biosensors can be configured to monitor invasively, minimally invasively, or non-invasively. The user devices discussed in connection with FIGS. 1-3E, as well as the methods discussed in greater detail below can be used with or include biosensors, hardware, patches, and/or wearables disclosed in U.S. Pat. Nos. 9,008,745; 9,182,368; 10,173,042; 10,595,754; U.S. application Ser. No. 15/876,678 (U.S. Pub. No. 2018/0140235); U.S. application Ser. No. 14/812,288 (U.S. Pub. No. 2016/0029931); U.S. application Ser. No. 14/812,302 (U.S. Pub. No. 2016/0029966); U.S. Pat. No. 10,820,860; U.S. application Ser. No. 16/888,105 (U.S. Pub. No. 2020/0375549); U.S. application Ser. No. 16/558,558 (U.S. Pub. No. 2020/0077931); and U.S. application Ser. No. 17/167,795 (U.S. Pub. No. 2021/0241916).

### Methods for Biomonitoring and Associated Biosensor Excitation Methods

In some embodiments, the biosensors described herein are configured to sense one or more analytes of interest in a body fluid by employing various sensing techniques, such as electrochemical sensing techniques (e.g., amperometric sensing, potentiometric sensing conductometric sensing, etc.) or other suitable sensing techniques. For example, as discussed above, a biosensor of the present technology can include (a) one or more electrodes (e.g., one or more working electrodes, one or more reference electrodes, and/or one or more counter electrodes), and (b) an electronics system operably connected to the one or more electrodes. The one or more electrodes can include or be formed at least in part by corresponding sets or arrays of one or more microneedles. The microneedles can be configured to penetrate, for example, the stratum corneum of a user's epidermis and to access a body fluid (e.g., interstitial fluid and/or blood) within the user's skin or subcutaneously. The electronics system can be configured to apply an excitation signal (also referred to herein as a “drive signal,” an “interrogation signal,” an “excitation voltage,” and/or a “bias voltage”) to working electrode(s) that differs from a signal applied to corresponding reference electrode(s), for example, to create a difference in potential between the working electrode(s) and the reference electrode(s). In turn, the electronics system can measure signals (e.g., voltages, currents, resistances, capacitances, impedances, gravimetric signals, and/or various other suitable signals) from the electrodes that are affected by—and therefore provide an indication of—concentrations of analytes in the body fluid of the user. Accordingly, the biosensor can (a) use the measured signals to determine concentrations of one or more analytes of interest in the body fluid of the user and/or (b) combine the concentration data with other information about the user to determine various health conditions of the user.

As discussed above, an excitation signal applied to a working electrode can often be a constant, DC signal. Thus, the biosensor can often be operated in a diffusion limited steady state as described by Equation 1 below:

I_{d}=nFAD(dC/dx) (1)

where I_{d} is diffusion limited current, n is a number of electrons transferred in the process, F is Faraday's constant, A is surface area of the working electrode, D is a diffusion coefficient of an analyte of interest, and (dC/dx) is a concentration gradient of the analyte of interest at the surface of the working electrode. Equation 1 above shows that the diffusion limited current I_{d} is proportional to the concentration of the analyte of interest but that properties of the biosensor and/or the working electrode (e.g., surface area, diffusion properties) must be known, assumed, or measured in order to calculate absolute concentrations of the analyte of interest. Such properties, however, are often difficult to measure or isolate from analyte concentration when the biosensor is in the steady state condition described by Equation 1 above. In addition, some or all of these properties (e.g., the diffusion properties immediately above or around the electrode and/or the functionalized detection-surface parameter of the electrode) may depend at least in part on manufacturing variability, how the biosensor is applied to the user's body, and/or other factors that can make it difficult to make accurate assumptions regarding these properties.

For example, as described above, the total surface area of a working electrode can differ from a total surface area of another working electrode (e.g., due to manufacturing variability). Additionally, or alternatively, if functionalized detection-surface parameter of a working electrode represents only 90% of the total surface area configured to detect an analyte of interest in a body fluid, the 10% decrease will affect the diffusion limited current observed at the working electrode but will be difficult to quantify and/or separate from analyte concentration contribution to the diffusion limited current. In addition, the functionalized detection-surface parameter of a working electrode can be largely unique to each use or application of the biosensor. The functionalized detection-surface parameter of a working electrode positioned within a user's body may also change over time (e.g., due to the biosensor device coming off of the user's skin or to an absence of a body fluid surrounding the working electrode). Therefore, estimating the effective surface area of a working electrode can be difficult and/or prone to error.

To address these concerns, biomonitoring systems of the present technology can apply excitation signals that include perturbations (e.g., time-varying characteristics, such as rising step changes) to one or more electrodes (e.g., one or more working electrodes, one or more reference electrodes, and/or one or more counter electrodes) of corresponding biosensors. The perturbations can (a) perturb the steady state operation of the corresponding biosensors and (b) cause one or more electrochemical reactions among adsorbed species in the user's body fluid and/or capacitive charging of the surface of one or more electrodes. In turn, the biomonitoring systems can use actual responses and modeled responses of the biosensors to the perturbations to determine one or more parameters or properties of the biomonitoring systems and/or the surrounding environment. Stated another way, as previously discussed, it may be difficult to isolate system-dependent parameters/properties from analyte concentration when the biosensor is operating in the steady state. Accordingly, a biomonitoring system of the present technology is configured to perturb the biosensor from the steady state via a time-varying excitation signal to allow system-dependent parameters/properties and/or parameters/properties of the environment surrounding the biosensor to be deconvoluted from analyte concentration in a response of the biosensor to the perturbation. In still other words, electrochemical reactions arising as a result of application of a perturbation can cause changes in response signals of electrodes of the biosensor, and these changes can be measured and analyzed in order to extract more information about the biosensor and/or the surrounding environment.

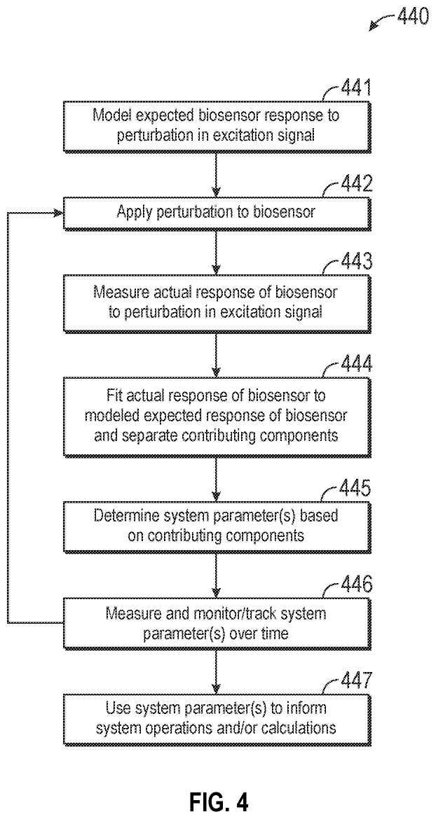

FIG. 4 is a flow diagram illustrating an example method 440 of determining biomonitoring system parameters using excitation signal perturbations in accordance with various embodiments of the present technology. The method 440 is illustrated as a set of steps or blocks 441-447. All or a subset of one or more of the blocks 441-447 can be executed by various components of a biomonitoring and healthcare guidance system (e.g., the biomonitoring and healthcare guidance system 102 of FIG. 1), by various components of one or more user devices (e.g., one or more of the user devices 104 of FIG. 1, such as one or more biosensors 104a, one or more mobile devices 104b, one or more wearable devices 104c), and/or by various components of biosensor devices (e.g., the biosensor devices 200 of FIGS. 2A and/or 2B, and/or the biosensor device 300 of FIGS. 3A-3E). As a specific example, all or a subset of one or more of the blocks 441-447 of the method 440 can be executed by various components of a biosensor, such as one or more electrodes (e.g., one or more working electrodes, one or more reference electrodes, and/or one or more counter electrodes); one or more microneedles; one or more sets of microneedles; one or more needles, and/or an electronics system operably connected to the one or more electrodes, one or more microneedles, one or more sets of microneedles, and/or one or more needles. In these and other embodiments, all or a subset of one or more of the blocks 441-447 of the method 440 can be executed by a user, patient, or operator of the biomonitoring and healthcare guidance system, of one or more of the user devices, and/or of the biosensor devices. In these and still other embodiments, any one or more of the blocks 441-447 can be executed in accordance with any portion of the discussion above, such all or a subset of the discussion of FIG. 1-3E above. Furthermore, all or a subset of one or more the blocks 441-447 can be performed before, during, or after a biosensor is applied to a user's body. As a specific example, block 441 can be executed before, during, or after a biosensor is applied to a user's body; and blocks 442-447 can be performed after the biosensor is applied to the user's body and/or in response to the application.

The method 440 begins at block 441 by modeling an expected response of a biosensor to a perturbation in an excitation signal applied to the biosensor. For example, as discussed above, an electronics system of the biosensor can be configured to apply an excitation voltage signal to a working electrode of the biosensor that differs from a voltage signal applied to a reference electrode of the biosensor. This can create a difference in potential between the working electrode and the reference electrode and/or can, if the difference in potential remains constant for a long enough period of time, result in the biosensor operating in a diffusion limited steady state described by Equation 1 (dC/dx) of the analyte of interest at the surface of the electrode). In turn, one or more of the isolated variables can be used to extract other information about the biosensor and/or the environment (e.g., interstitial fluid or another body fluid) surrounding the electrode or other components of the biosensor, as described in greater detail below with reference to blocks 442-447 of the method 440.

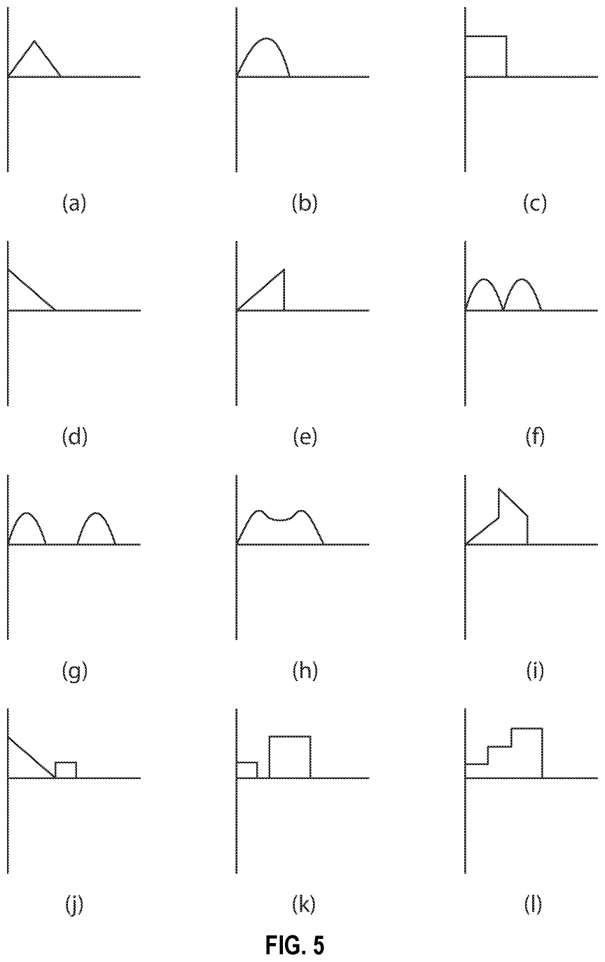

A perturbation can be a time-varying characteristic (e.g., a rising step change) of an excitation signal that results in or corresponds to a suitable excitation signal waveform applied to (e.g., the working electrode of) the biosensor. The excitation signal waveform can include one or more voltages, ramps, oscillations, and/or other properties. For example, FIG. 5 illustrates several suitable perturbations in excitation signals that can be used in various embodiments of the present technology. As shown in FIG. 5, perturbations of the present technology can be, include, or resemble a (a) a triangular wave (b) a sinusoidal wave, (c) square wave or pulse, (d) a sawtooth wave, (e) a ramp wave, (f) a wave resembling a full-wave rectified sinusoidal wave, (g) a wave resembling a half-wave rectified sinusoidal wave (or a sequence of two sinusoidal waves spaced apart in time), (h) a complex wave, an arbitrary wave (not shown), or any other suitable wave or portion thereof. In these and other embodiments, perturbations can be, include, or resemble multiple waves. For example, a perturbation can be, include, or resemble a mix of two or more waves (e.g., a triangular wave mixed with a square wave, as shown at (i) in FIG. 5). As another example, a perturbation can be, include, or resemble a sequence of two or more waves (e.g., a sawtooth wave followed immediately by a square wave with a different maximum amplitude, as shown at (j) in FIG. 5; or a sequence of amplitude-modulated square wave pulses spaced out in time). As still another example, a perturbation can be, include, or resemble a sequence of voltage steps (e.g., a stepped-up ramp wave, as shown at (1) in FIG. 5). Although each of the perturbations shown in FIG. 5 include only positive voltage components, other perturbations of the present technology can include negative voltage components in addition to or in lieu of positive voltage components.

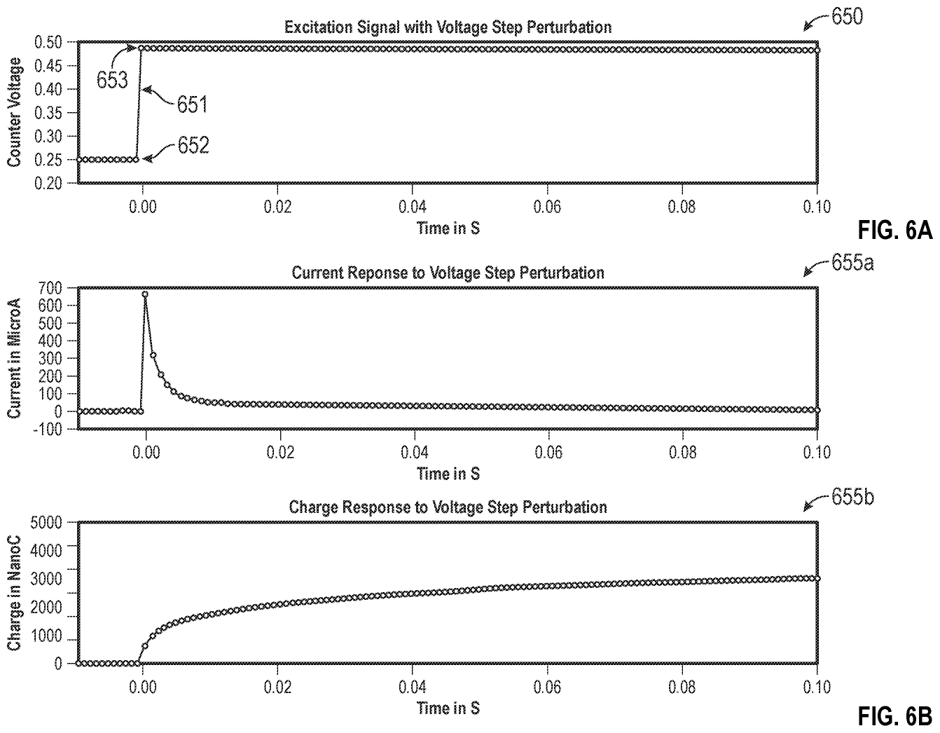

As a specific example, a perturbation in an excitation signal of the present technology is, includes, or resembles a square wave (e.g., a single step in potential, or an oscillating square wave potential with a predefined oscillation frequency between two potential levels) that can transition between two voltages (e.g., an “on” voltage and an “off” voltage). Continuing with this example, the perturbation can correspond to a rising edge, to a falling edge, or to both the rising and falling edges of the square wave. FIG. 6A illustrates a line plot 650 of an example of an exaction signal with such a perturbation 651. In particular, FIG. 6A illustrates that the perturbation 651 is, includes, or resembles a leading/rising edge of a square wave. More specifically, the perturbation 651 is, includes, or resembles a positive voltage step in poise potential in the excitation signal between an “off” voltage level 652 and an “on” voltage level 653.

In some embodiments, the potential difference between the “off” voltage 652 and the “on” voltage 653 (and/or various other characteristics of perturbations of the present technology) can be selected such that charged species (e.g., salts, ions, molecules, etc.) in the body fluid are not oxidized and/or reduced, or are oxidized and/or reduced to a negligible extent. In these and other embodiments, the potential difference between the “off” voltage 652 and the “on” voltage 653 (and/or various other characteristics of perturbations of the present technology) can be selected such that a measurable net flow of current occurs at the electrode (e.g., such that capacitive charging of the surface of the electrode occurs), for example, due to the charged species moving toward or away from the surface of the electrode to create or destroy a double layer charging effect. In these and still other embodiments, the potential difference between the “off” voltage 652 and the “on” voltage 653 (and/or various other characteristics of perturbations of the present technology) can be selected such that a measurable diffusion limited process for the faradaic response occurs at the electrode.

Continuing with the above example of a perturbation that is, includes, or resembles a square wave excitation signal waveform for the sake of clarity and understanding, shifting the excitation voltage from the “off” state/voltage to the “on” state/voltage (e.g., at the rising edge of the square wave) is expected to cause various effects, such as a reaction of adsorbed species at the electrode, capacitive charging of the surface of the electrode, a diffusion limited process for the faradaic response, background currents, oxidation or reduction of secondary species, and/or other processes or reactions. For example, FIG. 6B illustrates two plots 655a and 655b that illustrate a current response and a charge response, respectively, of the biosensor to the voltage step perturbation 531 in the excitation signal illustrated in FIG. 6A. Any one or more of the expected electrochemical reactions discussed above can contribute in varying degrees to the current response illustrated in the plot 655a and/or to the charge response illustrated in the plot 655b. All or a subset of the above expected electrochemical reactions at each voltage of the excitation signal and/or at the dynamic transition between the voltage levels of the voltage step perturbation can therefore be mathematically modeled for each biosensor and used to determine one or more parameters or properties of the biosensor, of the corresponding biomonitoring system, and/or of the surrounding environment.

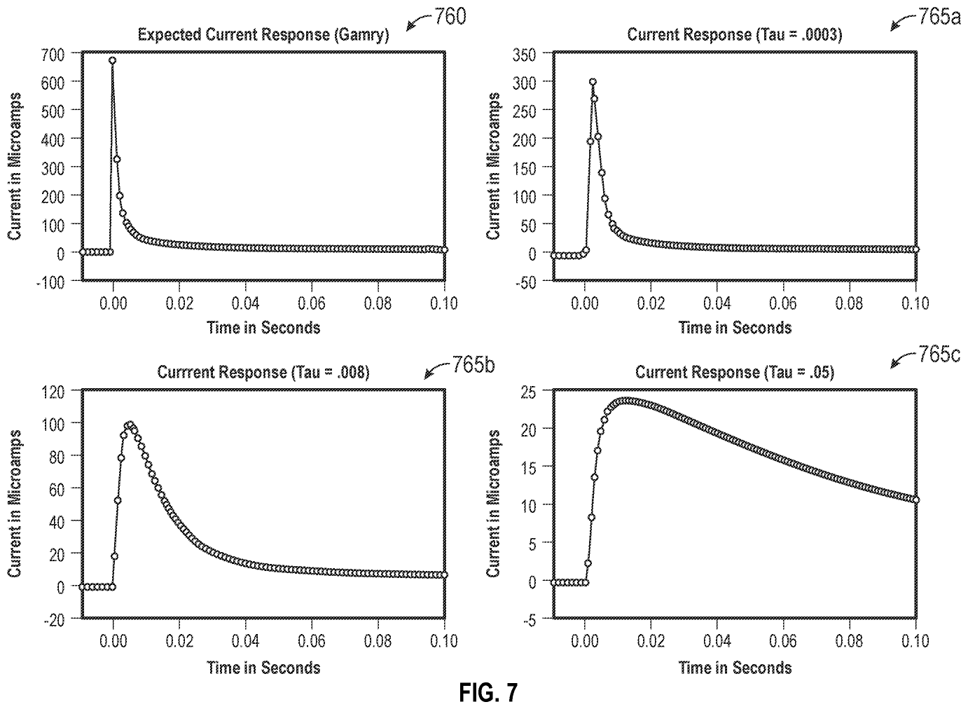

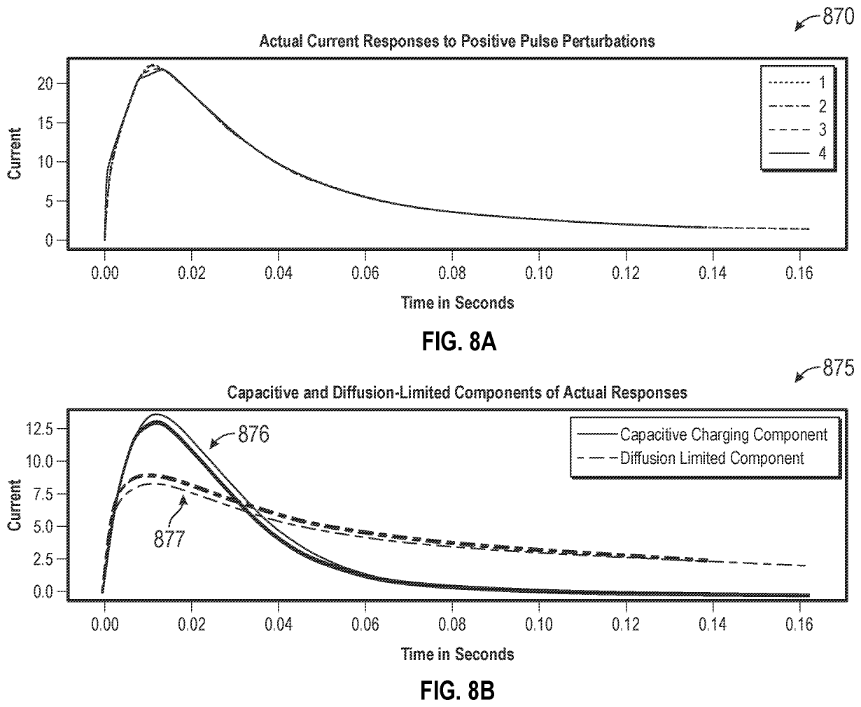

An example process that can be performed at block 441 of the method 440 of FIG. 4 for mathematically modeling an expected current response of a biosensor to one or more positive voltage step or square wave perturbations in an excitation signal applied to a working electrode of a biosensor will now be described in detail below with reference to FIGS. 7-8B and Equations 2-18. In some embodiments, a current response of a biosensor to a perturbation in an excitation voltage signal applied to a working electrode can be separated into three primary contributing components: a capacitive charging term i_{dl}, a diffusion limited term i_{diff}, and an adsorption term i_{ads}. These three contributing components can be modeled using Equation 2 below that describes expected current response as a function of time following an upward (or positive) voltage step perturbation in an excitation voltage signal applied to the working electrode of the biosensor:

$$i = {i}_{d l} + {i}_{diff} + {i}_{a d s} = \frac{E}{{R}_{s}} {e}^{\frac{- t}{{R}_{s} {C}_{d}}} + \frac{n F A C \sqrt{{D}_{0}}}{\sqrt{π t}} + {f}_{a d s} ( t ) \qquad ( 2 )$$

where n is the number of electrons transferred in the electrochemical process, F is Faraday's constant, A is the effective surface area or functionalized detection-surface parameter of the working electrode, D₀ is the diffusion constant of an analyte of interest, C is the concentration of the analyte of the analyte of interest, t is time, E is the potential difference of the voltage step in poise potential, Rₛ is the resistance of the solution or body fluid around the biosensor, C_{d} is the surface capacitance of the working electrode, and ƒ_{ads} (t) describes the immediate oxidation/reduction of any electroactive species that are adsorbed on the surface of the working electrode when the voltage step occurs.

In some embodiments, ƒ_{ads} (t) can be an impulse function (e.g., a Dirac Delta function). Therefore, assuming an infinite bandwidth, the expected current response can be modeled by:

$$\tilde{I} ( t ) = \{ \begin{matrix}{c}_{0} δ ( t ) + {c}_{1} {e}^{- t / {τ}_{1}} + {c}_{2} \frac{1}{\sqrt{t}} & t \geq 0 , & 0 < {τ}_{1} ⪡ 1 \\ 0 & t < 0 & \\\end{matrix} . \qquad ( 3 )$$

Due to (a) the low pass filter requirements or characteristics in an analog front end (AFE) of the electronics system of the biosensor, (b) bandwidth limitations, and/or (c) dynamic range limitations, a measured signal of the current response of the biosensor can be smoother in comparison to the expected current response signal provided by Equation 3 above. For example, FIG. 7 illustrates (a) a plot 760 of a modeled or expected current response signal provided by Equation 3 above, and (b) three other plots 765a-765c of smoother measured or actual current response signals of a biosensor corresponding to three different low-pass cutoffs.

The smoothed signals of the actual current responses illustrated in the plots 765a-765c (or other smoothed signals of actual current responses) can be modeled using a convolution function that takes the form of a first order low-pass filter having a low-pass cutoff corresponding to the characteristics or setup of a given biosensor. For example, Equations 4 and 5 below can be used to model a first order low pass filter to approximate the smoothing of the AFE in the electronics system of a given biosensor:

$$I ( t ) = {\int }_{- ∞}^{∞} h ( {t}^{′} ) \tilde{I} ( t - {t}^{′} ) {dt}^{′} \qquad ( 4 )$$

$$h ( t ) = \{ \begin{matrix}\frac{1}{{τ}_{h}} {e}^{- t / {τ}_{h}} & t \geq 0 \\ 0 & t < 0\end{matrix} . \qquad ( 5 )$$

In some embodiments, the impulse function ƒ_{ads} (t) of Equation 2 above (modeled by the Dirac Delta function in Equation 3 above) has a negligible contribution and/or a contribution that occurs too quickly for electronics of the biosensor to sample and measure. Accordingly, Equation 2 can be simplified to the following model provided by Equation 6 and can be used for a simple upwards step function perturbation in the excitation signal:

$$i = α {e}^{\frac{- t}{τ}} + \frac{β}{\sqrt{t}} \qquad ( 6 )$$

Equations 7-18 below outline how the above convolution integral from Equations 4 and 5 can be used to model an expected current response of the biosensor as measured downstream of the integrated analog filter. In the following, I₁(t) refers to the capacitive charging term i_{dl} described with reference to Equation 2 above, I₂ (t) refers to the diffusion limited term i_{diff} described with reference to Equation 2 above, and I_{D} (t) refers to the adsorption term i_{ads} described with reference to Equation 2 above.

Non-negative support for both Ĩ(t) and h(t) in Equations 3 and 5 above imply that the limits on the convolution integral of Equation 4 are finite and known, as shown in Equation 7 below:

I(t)=∫₀^{t}h(t′)Ĩ(t−t′)dt′ (7)

To derive a more explicit expression for I(t) than provided by Equation 7 above, the filter response to the adsorption input term (c₀δ(t)) of the unfiltered current Ĩ(t) provided by Equation 3 can be considered:

I_{D}(t)=c₀∫₀^{t}h(t′) δ(t−t′)dt′=c₀h(t) (8)

$$( {c}_{1} {e}^{\frac{- t}{{τ}_{1}}} )$$

The filter response I₂ (t) to the capacitive charging input term of the unfiltered current Ĩ(t) provided by Equation 3 can be considered:

$$\begin{matrix}{I}_{1} ( t ) = {c}_{1} {\int }_{0}^{t} h ( {t}^{′} ) {e}^{- ( t - {t}^{′} ) / {τ}_{1}} d {t}^{′} \\ = \frac{{c}_{1}}{{τ}_{h}} {\int }_{0}^{t} {e}^{- {t}^{′} / {τ}_{h}} {e}^{- ( t - {t}^{′} ) / {τ}_{1}} {dt}^{′} \\ = \frac{{c}_{1}}{{τ}_{h}} {e}^{- t / {τ}_{1}} {\int }_{0}^{t} {e}^{- {t}^{′} / {τ}_{h}} {e}^{{t}^{′} / {τ}_{1}} {dt}^{′} \\ = \frac{{c}_{1}}{{τ}_{h}} {e}^{- t / {τ}_{1}} {\int }_{0}^{t} {e}^{a {t}^{′}} {dt}^{′} \\ a = 1 / {τ}_{1} - 1 / {τ}_{h} \\ = \frac{{c}_{1}}{{τ}_{h}} {e}^{- t / {τ}_{1}} \frac{1}{a} ( {e}^{a t} - 1 ) \\ = \frac{{c}_{1}}{a {τ}_{h}} ( {e}^{- t / {τ}_{h}} - {e}^{- t / {τ}_{1}} )\end{matrix} \qquad ( 9 )$$

Now, when τ1«τₕ and t>τ₁:

$$a \approx 1 / {τ}_{1} , {e}^{- t / {τ}_{h}} ⪢ {e}^{- t / {τ}_{1}} ( t > {τ}_{1} ) ⇒ {I}_{1} ( t ) = \frac{{c}_{1} {τ}_{1}}{{τ}_{h}} {e}^{- t / {τ}_{h}} . \qquad ( 10 )$$

And the filter response to the diffusion limited input term

$$( {c}_{2} \frac{1}{\sqrt{t}} )$$

of the unfiltered current Ĩ(t) provided by Equation 3 is given by:

$${I}_{2} ( t ) = \frac{{c}_{2}}{{τ}_{h}} {\int }_{0}^{t} {e}^{- {t}^{′} / {τ}_{h}} \frac{1}{\sqrt{t - {t}^{′}}} d {t}^{′} \qquad ( 11 )$$

which can be solved by defining:

$$u = \sqrt{t - {t}^{′}} ⇒ d u = - \frac{1}{2} \frac{1}{\sqrt{t - {t}^{′}}} d {t}^{′} \qquad ( 12 )$$

implying:

$${I}_{2} ( t ) = - 2 \frac{{c}_{2}}{{τ}_{h}} {\int }_{\sqrt{t}}^{0} {e}^{- ( t - {u}^{2} ) / {τ}_{h}} du = 2 \frac{{c}_{2}}{{τ}_{h}} {e}^{- ( t - {u}^{2} ) / {τ}_{u}} {\int }_{0}^{\sqrt{t}} {e}^{{u}^{2} / {τ}_{h}} d u . \qquad ( 13 )$$

Equation 13 above can be further simplified using:

$$v = \frac{u}{\sqrt{{τ}_{h}}} ⇒ d v = \frac{d u}{\sqrt{{τ}_{h}}} ⇒ {\int }_{0}^{\sqrt{t}} {e}^{{u}^{2} / {τ}_{h}} du = \sqrt{{τ}_{h}} {\int }_{0}^{\sqrt{t / {τ}_{h}}} {e}^{{v}^{2}} d v . \qquad ( 14 )$$

And the integral of Equation 14 above can be expressed in terms of Dawson's integral (also known as Dawson's function or the imaginary error function):

$${D}_{+} ( x ) = {e}^{- {x}^{2}} {\int }_{0}^{x} {e}^{{y}^{2}} dy ⇒ {e}^{{x}^{2}} {D}_{+} ( x ) = {\int }_{0}^{x} {e}^{{y}^{2}} d y = \frac{\sqrt{π}}{2} erfi ( x ) ⇒ {\int }_{0}^{\sqrt{t}} {e}^{{u}^{2} / {τ}_{h}} du = \sqrt{{τ}_{h}} \frac{\sqrt{π}}{2} erfi ( \sqrt{t / {τ}_{h}} ) . \qquad ( 15 )$$

Accordingly, the filter response to the diffusion limited input term

$$( {c}_{2} \frac{1}{\sqrt{t}} )$$

of me unfiltered current Ĩ(t) provided by Equation 3 can be written as:

$${I}_{2} ( t ) = 2 \frac{{c}_{2}}{{τ}_{h}} {e}^{- t / {τ}_{h}} \sqrt{{τ}_{h}} \frac{\sqrt{π}}{2} erfi ( \sqrt{t / {τ}_{h}} ) = {c}_{2} \sqrt{\frac{π}{{τ}_{h}}} {e}^{- t / {τ}_{h}} erfi ( \sqrt{t / {τ}_{h}} ) \qquad ( 16 )$$

Therefore, the expected current response of the filter to the voltage step perturbation in the excitation signal applied to the working electrode of the biosensor, as measured downstream of the integrated analog filter, can be written as:

$$I ( t ) = {I}_{D} ( t ) + {I}_{1} ( t ) + {I}_{2} ( t ) = {c}_{0} h ( t ) + \frac{{c}_{1} {τ}_{1}}{{τ}_{h}} {e}^{- \frac{t}{{τ}_{h}}} + {c}_{2} \sqrt{\frac{π}{{τ}_{h}}} {e}^{\frac{- t}{{τ}_{h}}} erfi ( \sqrt{\frac{t}{{τ}_{h}}} ) \qquad ( 17 )$$

or, when the adsorption input term is considered negligible, as:

$$I ( t ) = \frac{{c}_{1} {τ}_{1}}{{τ}_{h}} {e}^{- \frac{t}{{τ}_{h}}} + {c}_{2} \sqrt{\frac{π}{{τ}_{h}}} {e}^{\frac{- t}{{τ}_{h}}} erfi ( \sqrt{\frac{t}{{τ}_{h}}} ) \qquad ( 18 )$$

Equations 8, 10, 16, 17, and/or 18 therefore mathematically model a relationship between (a) a current response of the biosensor to a rising step change in potential in the excitation signal applied to the biosensor, (b) the concentration of the analyte of interest, and/or (c) one or more system-dependent parameters. Furthermore, because each contributing component of the current response model described above has a linear and additive nature, additional parameters (e.g., used to model other parts of the biosensor system) can be easily added to the model in some embodiments. For example, resistance and/or impedance between a working electrode and a reference electrode can be added to and/or isolated from the model discussed above with reference to Equations 2-18.

Although block 441 of the method 440 is discussed above in the context of modeling an expected current response of a biosensor to a positive voltage step perturbation in an excitation signal applied to the biosensor, the method 440 is not so limited. For example, the method 440 can include modeling other types of biosensor response signals (e.g., charge, voltage, resistance, capacitance, impedance, and/or gravimetric response signals) that may similarly be affected by parameters/properties of the biosensor device and/or of the surrounding environment (e.g., surface area, diffusion properties, the quality of application of the biosensor to a user's body, manufacturing variation, etc.—also referred to herein as “system-dependent parameters”). The system-dependent parameters can be determined and/or isolated in accordance with the principles and techniques discussed above and/or that are included in the discussion of block 442-447 below. Additionally, or alternatively, different excitation signal waveforms, perturbations, potential differences between various voltage levels of the waveform, and/or current response models than described above with reference to Equations 2-18 can be used in other embodiments. For example, excitation signal waveforms, perturbations, potential differences between various voltage levels of the waveform, and/or current response models can be selected for use based on various factors, such as a power level or charge state of a battery or other power source of the biosensor device, a state (e.g., temperature, heart rate, state of exercise/activity) of the user, analyte(s) of interest, body fluid, and/or the occurrence of specific events (e.g., instructions received from another device in communication with the biosensor device). As a specific example, machine-learning techniques can be applied to select one or more characteristics of an excitation signal waveform or perturbation and/or to generate a model of a biosensor response (e.g., based at least in part on analyte(s) of interest and/or other events or factors). Suitable machine-learning techniques for use in the present technology are described in greater detail in U.S. application Ser. No. 16/558,558; U.S. application Ser. No. 17/167,795; U.S. application Ser. No. 17/236,753; and U.S. application Ser. No. 17/338,570, the disclosures of which are all incorporated by reference herein in their entireties. For example, machine-learning modules and engines can be used to calculate signal parameters based on health data, predictions of excitations, feature group generation/classification, etc. Model training can be performed using data disclosed herein.

Referring again to FIG. 4, the method 440 can continue at blocks 442 and 443 by applying the perturbation to (e.g., a working electrode or another electrode of) the biosensor and measuring an actual response of the biosensor to the perturbation. In some embodiments, the actual response can be an actual current response of the biosensor to the voltage step perturbation described above. FIG. 8A illustrates a line plot 870 showing four measured current responses of a biosensor in response to four perturbations (rising voltage steps) applied to a working electrode of the biosensor at four different times via an excitation signal.

At block 444, the method 440 of FIG. 4 can continue by fitting an actual response measured at block 443 to the model of an expected response determined, selected, or generated at block 441. In some embodiments, fitting an actual response to a model of an expected response can include fitting an actual current response of a biosensor to the model of an expected current response of the biosensor (such as the model provided by Equations 8, 10, 16, 17, and/or 18 above), and simultaneously solving for the capacitive charging and diffusion limited contributions to the measured current response. For example, FIG. 8B illustrates a line plot 875 showing capacitive charging components 876 and diffusion limited components 877 that each correspond to one of the actual current response signals illustrated in the plot 870 of FIG. 8A. More specifically, the capacitive charging component signal 876 and the diffusion limited component signal 877 of each actual current response signal illustrated in the plot 870 can be separated from one another by fitting the actual current response signal to the model provided by Equations 10, 16, and 18 above. In this manner, a model of an expected response of a biosensor determined, selected, or generated at block 441 of the method 440 of FIG. 4 can be used at block 444 to separate an actual response of the biosensor measured at block 443 into its individual contributing components.

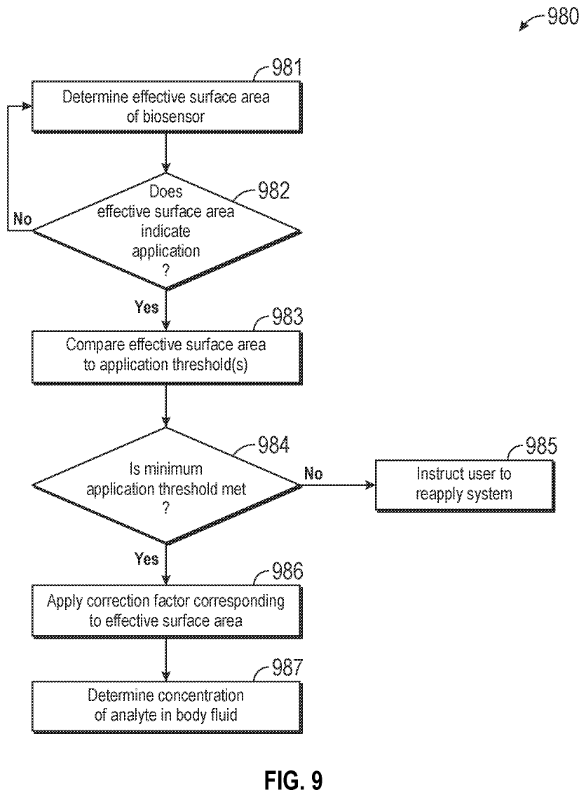

At block 445, the method 440 can continue by determining or extracting, based at least in part on one or more of the individual contributing components identified at block 444, system-dependent parameters or properties and/or information regarding the environment (e.g., interstitial fluid or another body fluid) surrounding the electrode or other components of the biosensor. For example, as discussed above, the surface of the electrode that was subjected to the perturbation at block 442 can be considered a capacitor with double-layer charging. Therefore, because capacitance of a parallel plate capacitor is tied to its surface area, the capacitive charging component (e.g., a capacitive charging component 876 in the line plot 875 of FIG. 8B) determined at block 444 of an actual current response of the biosensor measured at block 443 can be used to determine the effective surface area or functionalized detection-surface parameter of the electrode. The effective surface area or the functionalized detection-surface parameter of the electrode can be used to determine a total surface area of the working electrode resulting from manufacturing. Additionally, or alternatively, as discussed in greater detail below with reference to FIG. 9, the effective surface area or functionalized detection-surface parameter of the working electrode can be used as a proxy for determining the extent, quality, propriety, and/or success of application of the biosensor to a user's body.

The individual contributing components identified at block 444 can be used at block 445 to determine other information of the biomonitoring system and/or of the surrounding environment. For example, the diffusion limited component can be used to determine one or more diffusion properties of the biomonitoring system (e.g., diffusion properties adjacent or near the biosensor), such as a diffusion coefficient of an analyte of interest in a body fluid accessed by the biosensor. In some embodiments, the individual contributing components identified at block 444 can be used to determine absolute concentration of an analyte of interest (e.g., glucose) in a body fluid accessed by the biosensor. For example, the individual contributing components identified at block 444 can be used to determine the functionalized detection-surface parameter or effective surface area of an electrode and a diffusion coefficient of an analyte of interest that, in turn, can be used in Equation 1 above to determine or provide an indication of an absolute concentration of the analyte of interest in a body fluid accessed by the electrode. In these and other embodiments in which the model includes components describing the resistance/impedance relationship between a working electrode and a reference electrode, these components can be used to determine the resistance or impedance between the working electrode and the reference electrode that may be affected by (and therefore provide an indication of), for example, properties of the user's interstitial fluid (or other body fluid) and/or local tissue (e.g., the user's epidermis).

At block 446, the method 440 can, optionally, continue by measuring, tracking, and/or monitoring one or more of the parameters/properties determined at block 445 overtime. For example, as shown in FIG. 4, the method 440 can return to block 442 (e.g., after any one of blocks 443-446) to apply another perturbation to the biosensor and then proceed to (a) measure a resulting response of the biosensor (block 443), (b) fit the response to the model to separate the response into its individual contributing components (block 444), and (c) determine one or more system-dependent parameters and/or properties of the surrounding environment (block 445). In some embodiments, a biosensor device can be configured to apply a perturbation to the biosensor at predetermined time intervals (e.g., once every minute, 2 minutes, 5 minutes, 10 minutes, 15 minutes, 20 minutes, 30 minutes, 60 minutes, 2 hours, etc.). In these and other embodiments, the time interval and/or a determination to apply a perturbation to the biosensor can be based at least in part on detection of the occurrence of certain events, such as a detected change in a system parameter (e.g., determined by another sensor of the biomonitoring system), receipt of instructions from another device (e.g., a mobile device, a wearable device, a biomonitoring and healthcare guidance system) in communication with the biosensor device, and/or a state of the user (e.g., a temperature of the user, an activity (resting, exercising showering, etc.) of the user). In these and still other embodiments, the time interval and/or a determination to apply a perturbation to the biosensor can be based at least in part on a power level or charge state of a battery or other power source of the biosensor device. In some embodiments, the time interval between perturbations can vary (e.g., due to optimization or updates, such as from machine-learning techniques as additional data from the user and/or other users becomes available and is processed/analyzed).

The method 440 (at block 446) can use several measurements taken over time to detect the occurrence of one or more events and/or changes in the system-dependent parameters and/or in the properties of the surrounding environment by, for example, (a) comparing measurements taken later in time to measurements to previous measurements and/or (b) comparing measurements to various other corresponding baselines or thresholds. More specifically, taking effective surface area or functionalized detection-surface parameter of an electrode of a biosensor as a specific example, the method 440 can determine a first effective surface area or first functionalized detection-surface parameter of the electrode (e.g., during a first iteration through all or a subset of blocks 441-445 of the method 440, and/or when the biosensor is initially applied to a user's body) that can be used as a baseline effective surface area or functionalized detection-surface parameter for future measurements. In these embodiments, the method 440 can monitor the effective surface area/functionalized detection-surface parameter of the electrode over time in comparison to the first effective surface area/first functionalized detection-surface parameter and/or in comparison to one or more previously measured effective surface areas/functionalized detection-surface parameters of the electrode to detect changes in the effective surface area/functionalized detection-surface parameter of the electrode over time. If the method 440 determines that the effective surface area/functionalized detection-surface parameter of an electrode has decreased over time, the method 440 can determine that one or more microneedles forming the electrode may have slipped at least partially out of the user's skin and/or that at least some of the microneedles are no longer accessing a body fluid within the user's skin (e.g., due to the biosensor device falling or coming off of the user's skin). Similarly, if the method 440 determines that an effective surface area/functionalized detection-surface parameter of the electrode has increased over time, the method 440 can determine that insertion depth of one or more microneedles forming the electrode may have improved and/or a greater number of the microneedles have accessed the body fluid within the user's skin.

As another example, the method 440 can monitor the diffusion limited contribution component of current responses of the biosensor to perturbations over time to detect changes in the diffusion properties of the biosensor device and/or the surrounding environment. For example, before a biosensor is applied to a user's body, a membrane (e.g., a selective transport membrane) of the biosensor that is positioned at or proximate one or more electrodes of the biosensor and/or that is configured to interact with one or more analytes of interest in a body fluid can be dry. When the biosensor is applied to the user's body such that the membrane accesses a body fluid, the membrane begins to hydrate. As the membrane hydrates, diffusion properties of the membrane can change. Therefore, the method 440 can monitor the diffusion limited contribution component of the current response of the biosensor to perturbations over time to detect changes that can serve as a proxy for determining hydration state of the membrane.

Diffusion properties of the membrane can also change depending at least in part on potential hydrogen (pH) of analyte concentrations in the body fluid and/or biofouling, either or which can be unique to or dependent upon the user's physiology. Thus, the method 440 can monitor the diffusion limited contribution component of the current response of the biosensor to perturbations over time (e.g., once the membrane reaches a fully hydrated state) to detect changes indicative of changes in the user's physiology.

Diffusion properties can also change with changes in the user's tissue. For example, as a user's skin heals after being punctured with microneedles of the biosensor, diffusion can decrease and/or the recovery time for the biosensor to return to the steady state condition after being subjected to a perturbation in the excitation signal can change. Thus, the method 440 can monitor the diffusion limited contribution component and/or the current response over time and can use detected changes as a proxy to determine the extent of healing (e.g., of the user's skin) proximate the corresponding electrodes.

Furthermore, an amount of interstitial fluid within a user's skin can depend at least in part on a hydration level of the user. Thus, the method 440 can monitor the diffusion limited contribution of the current response over time and can use detected changes in diffusion properties as a proxy for hydration level of the user. For example, a decrease in diffusion can indicate the user is dehydrated or less hydrated than at a previous time when diffusion was higher.

As discussed above, properties of a user's body fluid and/or tissue local the biosensor can affect resistance or impedance measurements between a working electrode and a corresponding reference electrode. As such, the method 440 can monitor the resistance or impedance between the working electrode and the corresponding reference electrode overtime, and changes detected in these measurements can be used as a proxy to determining changes in properties of the user's body fluid and/or local tissue.

At block 447, the method 440 can continue by using the one or more parameters/properties determined at block 445 and/or tracked/monitored over time at block 446 to inform various system operations and/or calculations. For example, FIG. 9 is a flow diagram illustrating a method 980 for using effective surface areas/functionalized detection-surface parameters of electrodes of a biosensor for determining extent, quality, propriety, and/or success of application of the biosensor to a user's body. The method 980 can begin at block 981 by determining the effective surface area(s) of one or more electrodes (e.g., one or more working electrodes, one or more reference electrodes, and/or one or more counter electrodes) of a biosensor. In some embodiments, the effective surface area(s) can be determined in accordance with the discussion above with reference to block 445 of the method 440. In these and other embodiments, determining the effective surface area(s) can be performed before, during, and/or after the biosensor is applied to a user's body. For example, block 981 of the method 980 can be performed while the biosensor is submerged in a bath (e.g., to determine 100% effective surface area value(s) for one or more electrodes of the biosensor, which can be used to account for manufacturing variabilities and/or provide a baseline representing 100% or complete application of the biosensor to a user's body). As another example, block 981 of the method 980 can be performed as part of a process (e.g., in combination with block 982) for detecting application of the biosensor in a user's body and/or can be performed in response to an indication (e.g., from another device or sensor) that the biosensor has been applied to the user's body.

At block 982, the method 980 can continue by determining whether the effective surface area(s) of the electrode(s) indicate that the biosensor has been applied to a user's body. For example, if the effective surface area(s) of several electrodes of the biosensor are zero or negligible, the method 980 can determine that the biosensor is not applied to a user's body and can return to block 981. As another example, if the effective surface area(s) of all or nearly all of the electrodes of the biosensor are equivalent to the 100% application values discussed above, this can suggest that the biosensor is submerged in a bath instead of applied to a user's body. As such, the method 980 may return to block 981 in this scenario. On the other hand, if the effective surface area(s) of the electrode(s) suggest that the biosensor has been applied to a user's body, the method 980 can proceed to block 983.

At block 983, the method 980 can continue by comparing the effective surface area(s) determined at block 981 to one or more application thresholds. In some embodiments, the application thresholds can include one or more thresholds that apply to individual electrodes of the biosensor. For example, the application thresholds can include a threshold representing a minimum effective surface area of an electrode required to determine system-dependent parameters and/or properties of the environment surrounding the electrode. As another example, in embodiments in which a set or array of microneedles forms an electrode positioned at distal end regions of the microneedles, the application thresholds can include a threshold representing a minimum number of the microneedles corresponding to an electrode and that have distal end regions that are positioned within tissue and/or that access a body fluid of interest (e.g., interstitial fluid, blood). In some embodiments, the method 980 can deduce or determine the number of microneedles of an electrode having distal end regions that are positioned within tissue and/or that access a body fluid from the effective surface area of that electrode determined at block 981.

In these and other embodiments, the application thresholds can include one or more thresholds that apply to multiple electrodes of the biosensor in combination. For example, the application thresholds can include a threshold representing a minimum number of electrodes of the biosensor having effective surface areas greater than the minimum effective surface area threshold for a single electrode. As another example, the application thresholds can include a threshold representing a minimum number of microneedles (e.g., across multiple electrodes, such as across multiple working electrodes or across a working electrode and a reference electrode) having distal end regions that are positioned within tissue and/or that access a body fluid of interest.

As discussed in greater detail below with reference to block 986, the application thresholds can include thresholds that correspond to various correction factors that can be applied by the biosensor device based at least in part on the effective surface area of one or more of the electrodes of the biosensor. For example, multiple application thresholds can be used to define various ranges of application based on the effective surface area(s) of the electrode(s). Continuing with this example, the effective surface area(s) determined at block 981 can be individually or aggregately compared to the application thresholds, and one or more correction factors that correspond to the range(s) within which the effective surface area(s) fall can be applied to output signals of the biosensor. Additionally, or alternatively, one or more of these comparisons can be used to set or adjust other parameters of the biosensor device, such as a drive signal applied to one or more of the working electrode(s) of the biosensor.

At block 984, the method 980 can continue by determining whether a minimum application threshold is met by the biosensor. The minimum application threshold can represent a threshold below which the biosensor device is not able to meaningfully or accurately determine system-dependent parameters, properties of the environment surrounding the biosensor, analyte concentrations, and/or other information or parameters. In other words, the minimum application threshold can represent an extent or quality of application of the biosensor to the user's body below which the biosensor device is unlikely to (a) provide the user meaningful or accurate information and/or (b) function as intended, and at which the user should reapply the biosensor (or another biosensor) to the user's body. In some embodiments, the minimum application threshold can include any one or more of the thresholds discussed above with reference to block 983 and/or one or more other suitable thresholds. In these and other embodiments, whether the minimum application threshold is met can depend at least in part on the effective surface area(s) of the electrode(s).

If the minimum application threshold is not met at block 984, the method 980 can proceed to block 985 to instruct the user to reapply the biosensor to the user's body. Otherwise, the method 980 can proceed to block 986.

At block 986, the method 980 can continue by applying an appropriate correction factor, for example, to signals output from one or more electrodes of the biosensor. For example, as discussed above, the method 980 can apply a correction factor corresponding to an effective surface area of an individual electrode or an aggregate effective surface area of a combination of two or more electrodes. As another example, the method 980 can apply a correction factor to an electrode (e.g., positioned shallower in the user's skin and/or accessing a lesser amount of body fluid than another electrode) based at least in part on properties of the environment (e.g., the body fluid and/or tissue) surrounding the other electrode, the effective surface area of either or both electrodes, and/or a correction factor applied to the other electrode.

At block 987, the method 980 can continue by (a) detecting and/or determining concentrations of one or more analytes of interest in the body fluid, and/or (b) performing various other operations, based at least in part on the correction factor(s) applied at block 986. In some embodiments, detecting and/or determining the concentrations of the one or more analytes of interest, can include determining absolute concentrations of the one or more analytes of interest in accordance with the discussion of above with reference to the method 440 of FIG. 4.

Although the steps of the method 980 are discussed and illustrated in a particular order, the method 980 of FIG. 9 is not so limited. In other embodiments, the steps of the method 980 can be performed in a different order. In these and other embodiments, any of the steps of the method 980 can be performed before, during, and/or after any of the other steps of the method 980. Furthermore, a person skilled in the art will readily recognize that the method 980 can be altered and still remain within these and other embodiments of the present technology. For example, one or more steps (e.g., blocks 881, 882, 886, and/or 887) of the method 980 can be omitted and/or repeated in some embodiments.

Referring again to block 447 of the method 440 of FIG. 4, any of the other system-dependent parameters and/or determined properties of the environment surrounding the biosensor can be used to calibrate or otherwise control operation of the biosensor device. For example, one or more of the determined system-dependent parameters (e.g., effective surface area(s) of one or more electrode(s), hydration state of the membrane, etc.) and/or one or more properties of the environment surrounding the biosensor (e.g., pH or physiological makeup of a body fluid, resistance of the body fluid, hydration level of the user, extent of healing of tissue surrounding the biosensor, etc.) can be used to (a) select or calibrate one or more drive signals applied to one or more electrodes of the biosensor and/or (b) select or calibrate one or more correction factors applied to signals output from the one or more electrodes. Additionally, or alternatively, detected changes in the system-dependent parameters and/or pieces of information over time can be used to perform drift correction, adjust calibration parameters, and/or otherwise modify operation of the biosensor.

Although the steps of the method 440 are discussed and illustrated in a particular order, the method 440 of FIG. 4 is not so limited. In other embodiments, the steps of the method 440 can be performed in a different order. In these and other embodiments, any of the steps of the method 440 can be performed before, during, and/or after any of the other steps of the method 440. Furthermore, a person skilled in the art will readily recognize that the method 440 can be altered and still remain within these and other embodiments of the present technology. For example, one or more steps (e.g., blocks 446) of the method 440 can be omitted and/or repeated in some embodiments.

As discussed above, the techniques described herein can be adapted for use with multiple types of excitation waveforms (e.g., at least two, three, four, or five different waveforms). The waveforms can have different characteristics, such as shape, amplitude, frequency, maximum voltage, minimum voltage, etc. In such embodiments, the method can include selecting an excitation waveform based on various factors, such as the particular analyte(s) of interest and/or the detection routine to be performed by the biosensor. For example, a first waveform can be applied when measuring a concentration of a first analyte, a second waveform can be applied when measuring a concentration of a second analyte, and so on. The different waveforms can be sequentially applied to the same set or array of microneedles. Additionally, or alternatively, different waveforms can be concurrently applied to different sets or arrays of microneedles. In these and other embodiments, a type of signal processing (e.g., filtering) used for the biosensor response can be selected based on the analyte(s) of interest, the excitation waveform, the detection algorithm, and/or other factors. The method can optionally include obtaining multiple processed signals, each signal corresponding to a different excitation waveform and/or set of signal processing parameters. The processed signals can be individually or collectively analyzed to determine the concentrations of one or more different analytes.

In some embodiments, the present technology can be used to allow for multiplexed detection of multiple analytes on the same electrode, for example, by analyzing the transient response between two or more different potentials. For example, the techniques described herein can be adapted to model the effects of concentrations of multiple electrochemical species on a single working electrode. Subsequently, the model can be applied to deconvolute and/or otherwise isolate the individual concentration of each species. Optionally, machine learning-based techniques can also be applied to generate a model of the biosensor response to different analytes and/or determine the individual concentrations of different analytes.

Although primarily discussed above in the context of modeling a current response of a biosensor to a voltage step perturbation applied to an electrode of the biosensor (as measured downstream from a low pass filter), the present technology can select or use other models, perturbations, drive signals, correction factors, responses, and/or system parameters (e.g., intervals between application of perturbations to an electrode of a biosensor, a sequence of perturbations applied, etc.) in other embodiments. For example, a model, perturbation, drive signal, correction factor, analyzed biosensor response, and/or various other system parameters can be selected and/or based at least in part on a power level or charge state of a battery or other power source of a biosensor device. The model, perturbation, drive signal, analyzed response, correction factor, and/or system parameters can additionally, or alternatively, can be selected and/or based at least in part on detection of the occurrence of particular events (e.g., detection of a user's state of exercise, heart rate, skin temperature, etc.) or on user settings. As still another example, the model, perturbation, drive signal, correction factor, analyzed response, and/or system parameters can be selected and/or based at least in part on manufacturing-related inputs/data/parameters, such as manufacturing-related calibration data as disclosed in greater detail in U.S. application Ser. No. 17/236,753 (U.S. Pub. No. 2021/0321942) and U.S. application Ser. No. 17/578,386. Continuing with this example, the model, perturbation, drive signal, correction factor, analyzed response, and/or system parameters can be selected and/or based at least in part on manufacturing-related inputs/data/parameters (e.g., a first model, a first perturbation, a first drive signal, a first correction factor, a first analyzed response, and/or first system parameters can be selected for use with electrodes of a biosensor corresponding to an inner substrate or wafer; and another model, another perturbation, another drive signal, another correction factor, another analyzed response, and/or other system parameters can be selected for use with electrodes of a biosensor corresponding to an outer substrate or wafer), for example, to account for manufacturing variability. As a specific example, a first drive signal used for electrodes corresponding to an inner wafer can have a first shape/waveform/perturbation and a second drive signal used for electrodes corresponding to an outer wafer can have the first shape/waveform/perturbation but with different or scaled voltage amplitudes and/or voltage step levels.

In these and other embodiments, models, excitation signals, perturbations, correction factors, analyzed responses, and/or system parameters can be updated, adjusted, and/or retrained over time. For example, a biosensor device can be configured to (e.g., wirelessly) receive programs and/or updates for models, excitation signals, perturbations, correction factors, analyzed responses, and/or other system parameters; and to accordingly adjust models, excitation signals, perturbations, correction factors, analyzed responses, and/or other system parameters employed by the biosensor device. As another example, models, excitation signals, perturbations, correction factors, analyzed responses, and/or system parameters can be updated, adjusted, and/or retrained based at least in part on detection of the occurrence of specific events (e.g., detection of a user's activity, heart rate, skin temperature, etc.), on a defined schedule (e.g., every minute, every hour, every day, every few days, every week, etc.), on user-defined settings, etc. As still another example, models, excitation signals, perturbations, correction factors, analyzed responses, and/or system parameters can be updated, adjusted, and/or retrained based at least in part on availability of additional data or information. As a specific example, knowledge of a user's glucose levels (e.g., from measurements taken by the biosensor device or another sensor) can be used to simplify the model (e.g., by inserting a known glucose concentration value into the model) to update or retrain the model, and/or to more accurately determine one or more other variables of the model and any other system-dependent parameters or properties of the surrounding environment that are determined based at least in part on the variables.

In some embodiments, models, excitation signals, perturbations, correction factors, analyzed responses, and/or system parameters can be updated, adjusted, and/or retrained over time using machine learning engines or techniques. For example, a biosensor device of the present technology can be configured to use an independent and/or unique drive signals for each electrode and/or each biosensor of the device, and the individual drive signals can be separately calibrated with machine learning using user-specific data. As a specific example, a first electrode can receive a first drive signal that is calibrated based at least in part on properties of the environment (e.g., body fluid, tissue) surrounding a distal tip region of a microneedle of the first electrode that is configured to sense a first analyte, and a second electrode can receive a second drive signal that is calibrated based at least in part on properties of the environment (e.g., body fluid, tissue) surrounding a distal tip region of a microneedle of the second electrode that is configured to sense a second analyte. The first drive signal and the second drive signal can be calibrated at the same time or at different times.

As another specific example, the drive signal of one electrode can be calibrated and/or adjusted based on parameters/properties determined at another electrode. For example, one electrode configured to detect glucose levels can receive a first drive signal and another electrode configured to detect ketone levels can receive a second drive signal. The first drive signal of the one electrode can be based, calibrated, and/or adjusted at least in part on ketone levels detected at the other electrode. The drive signals of either electrode can be based, calibrated, and/or adjusted at least in part on one or more other parameters/properties (e.g., skin temperature, analytes levels, motions, etc.) detected at the other electrode.

### EXAMPLES

Several aspects of the present technology are set forth in the following examples. Although several aspects of the present technology are set forth in examples specifically directed to methods, systems, and computer-readable mediums; any of these aspects of the present technology can similarly be set forth in examples directed to any of devices/apparatuses, systems, methods, and computer-readable mediums in other embodiments. Similarly, any of the methods set forth in the following examples can be incorporated into any of the devices and systems described above.
1. A method of operating a biosensor device, the method comprising:
applying an excitation signal to a biosensor, wherein the biosensor includes a plurality of electrodes positionable within a user's skin to access interstitial fluid therein, wherein an electrode of the plurality of electrodes is configured to detect a presence of an analyte of interest in the interstitial fluid, wherein applying the excitation signal includes applying the excitation signal to the electrode, and wherein the excitation signal includes a time-varying characteristic configured to perturb a diffusion limited steady state of the biosensor;
measuring a response of the biosensor to the time-varying characteristic, wherein the response of the biosensor includes a first contribution that depends at least in part on capacitive charging of a surface of the electrode and a second contribution that depends at least in part on a diffusion limited process for a faradaic response at the electrode;
separating the first contribution from the second contribution; and
determining, based at least in part on the separated first contribution or the separated second contribution, at least one operational parameter that includes:
   (a) one or more system-dependent parameters of the biosensor device,
   (b) one or more properties of the interstitial fluid surrounding the electrode,
   (c) one or more properties of tissue surrounding the electrode, or
   (d) any combination thereof.
2. The method of example 1, wherein:
the at least one operational parameter includes an effective surface area of the electrode; and
the method further comprises detecting application of the biosensor device to the user's body based at least in part on the effective surface area of the electrode.
3. The method of example 1 or example 2, further comprising controlling operation of the biosensor based at least in part on the determined at least one operational parameter to increase detection accuracy of the biosensor for the analyte of interest.
4. The method of any of examples 1-3, further comprising:
periodically applying the time-varying characteristic to the electrode and monitoring the response of the biosensor; and adjusting operation of the biosensor based at least in part on changes detected in the response of the biosensor over time.
5. The method of example 4, wherein the changes correspond to changes at a detection site of the electrode within the user's skin.
6. The method of example 4 or example 5, wherein adjusting the operation of the biosensor includes:
performing drift correction or calibration based at least in part on the detected changes;
adjusting the operation of the biosensor according to the drift correction or the calibration to generate a detection output for the analyte of interest; and
determining a concentration of the analyte of interest based on the detection output.
7. The method of any of examples 1-6, wherein the response of the biosensor is a current response of the biosensor.
8. The method of any of examples 1-7, wherein the time-varying characteristic includes a positive voltage step in the excitation signal.
9. The method of any of examples 1-8, wherein:
the excitation signal includes a square wave, a triangular wave, a sawtooth wave, or any combination thereof; and
the time-varying characteristic includes all or a portion of the square wave, the triangular wave, the sawtooth wave, or any combination thereof.
10. The method of any of examples 1-9, wherein the response of the biosensor includes a third contribution that depends at least in part on oxidation/reduction of electroactive species in the interstitial fluid that are adsorbed on the surface of the electrode.
11. The method of any of examples 1-10, wherein separating the first contribution from the second contribution includes fitting the response of the biosensor to a model of an expected response of the biosensor to the time-varying characteristic.
12. The method of example 11, wherein fitting the response of the biosensor to the model includes simultaneously solving for (a) the first contribution and the second contribution or (b) the first contribution, the second contribution, and the third contribution.
13. The method of example 11 or example 12, wherein the model of the expected response is a model of an expected current response of the biosensor to the time-varying characteristic as measured downstream from an integrated analog filter.
14. The method of any of examples 11-13, further comprising modeling the expected response of the biosensor to the time-varying characteristic.
15. The method of any of examples 1-14, wherein:
the one or more system-dependent parameters includes an effective surface area of the electrode; and the determining includes determining the effective surface area from the separated first contribution.
16. The method of example 15, further comprising determining an absolute concentration of the analyte of interest based at least in part on the effective surface area of the electrode.
17. The method of any of examples 1-16, wherein:
the time-varying characteristic is a first time-varying characteristic, and the response is a first response;
the method further comprises monitoring the at least one operational parameter overtime; and
the monitoring includes:
   redetermining, based at least in part on a second response of the biosensor to a second time-varying characteristic in the excitation signal, the at least one operational parameter; and
   comparing the at least one operational parameter determined from the second response to the at least one operational parameter determined from the first response.
18. The method of example 17, wherein:
the at least one operational parameter determined from the first response include a first effective surface area of the electrode;
the at least one operational parameter determined from the second response include a second effective surface area of the electrode; and
the monitoring further includes determining a position of the electrode within the user's skin has changed based at least in part on a difference between the first effective surface area and the second effective surface area.
19. The method of example 17 or example 18, wherein:
the at least one operational parameter determined from the first response include first diffusion properties at a membrane proximate the electrode;
the at least one operational parameter determined from the second response include second diffusion properties at the membrane; and
the monitoring further includes determining a hydration state of the membrane based at least in part on a difference between the first diffusion properties and the second diffusion properties.
20. The method of any of examples 17-19, wherein:
the at least one operational parameter determined from the first response include first diffusion properties of the tissue;
the at least one operational parameter determined from the second response include second diffusion properties of the tissue; and the monitoring further includes determining an extent of healing of the tissue surrounding the electrode based at least in part on a difference between the first diffusion properties and the second diffusion properties.
21. The method of any of examples 17-20, wherein:
the at least one operational parameter determined from the first response include first diffusion properties of the interstitial fluid and/or the tissue;
the at least one operational parameter determined from the second response include second diffusion properties of the interstitial fluid and/or the tissue; and
the monitoring further includes determining a physiology or hydration level of the user has changed based at least in part on a difference between the first diffusion properties and the second diffusion properties.
22. The method of any of examples 1-21, wherein the excitation signal applied to the biosensor depends at least in part on a power usage protocol of the biosensor device, a charge state of a battery of the biosensor device, or a combination thereof.
23. The method of any of examples 1-22, wherein:
the excitation signal depends at least in part on detection of an occurrence of one or more events, on a temperature of the user, on a heart rate of the user, on a body fluid, or on any combination thereof; and
the one or more events include a detection of the user exercising, a determination that a specified amount of time has elapsed since a last perturbation of the diffusion limited steady state of the biosensor, or a combination thereof.
24. The method of any of examples 1-23, wherein:
the excitation signal corresponds to the analyte of interest;
the electrode is further configured to detect presence of another analyte of interest in the interstitial fluid;
the method further comprises applying another excitation signal to the biosensor; and
the other excitation signal corresponds to the other analyte of interest and includes a different time-varying characteristic configured to perturb the diffusion limited steady state of the biosensor.
25. The method of any of examples 1-24, wherein:
the electrode is a first electrode;
the excitation signal is a first excitation signal;
the method further comprises applying a second excitation signal to a second electrode of the plurality of electrodes; and
the second excitation signal includes a second time-varying characteristic configured to perturb the diffusion limited steady state of the biosensor.
26. The method of example 25, wherein:
the analyte of interest is a first analyte of interest;
the first excitation signal corresponds to the first analyte of interest;
the second electrode is configured to detect a presence of a second analyte of interest in the interstitial fluid; and
the second excitation signal corresponds to the second analyte of interest.
27. The method of example 26, wherein the first excitation signal is based at least in part on a concentration of the second analyte in the interstitial fluid determined based at least in part on the application of the second excitation signal to the second electrode.
28. A method, comprising:
determining an effective surface area of an electrode of a biosensor,
   wherein the biosensor is configured to determine a concentration of an analyte of interest in interstitial fluid of a user,
   wherein the electrode of the biosensor is positionable within tissue of the user to access the interstitial fluid when the biosensor is applied to the user's body, and
   wherein determining the effective surface area of the electrode includes determining the effective surface area from capacitive charging of a surface of the electrode that is measured when a perturbation in an excitation signal is applied to the electrode; and
detecting application of the biosensor to the user's body based at least in part on the determined effective surface area of the electrode.
29. The method of example 28, further comprising determining whether a minimum application threshold is met based at least in part on the determined effective surface area of the electrode.
30. The method of example 29, wherein the minimum application threshold includes:
a threshold representing a minimum effective surface area of the electrode for determining a concentration of the analyte of interest in the interstitial fluid; or
a threshold representing a minimum number of microneedles forming the electrode and that have distal end regions accessing the interstitial fluid.
31. The method of example 29, wherein the minimum application threshold includes:
a threshold representing a minimum aggregate effective surface area of effective surface areas of multiple electrodes of the biosensor;
a threshold representing a minimum number of electrodes of the biosensor meeting a minimum effective surface area for determining a concentration of the analyte of interest in the interstitial fluid;
a threshold representing an aggregate minimum number of microneedles forming two or more electrodes and that have distal end regions accessing the interstitial fluid; or
a threshold representing a minimum number of the electrodes of the biosensor having a minimum number of microneedles with distal end regions accessing the interstitial fluid.
32. The method of any of examples 29-31, further comprising instructing the user to reapply the biosensor to the user's body based at least in part on a determination that the minimum application threshold is not met.
33. The method of any of examples 28-32, further comprising applying a correction factor to signals generated at least in part by the electrode, wherein the correction factor is based at least in part on the determined effective surface area of the electrode.
34. The method of any of examples 28-33, further comprising adjusting the excitation signal based at least in part on the determined effective surface area of the electrode.
35. A method, comprising:
measuring a signal output from an electrode of a biosensor, wherein the signal is output in response to a perturbation in a drive signal that is applied to the electrode, wherein the electrode is positionable within tissue of a user to access a body fluid of the user and is configured to detect presence of an analyte of interest in the body fluid, and wherein the signal output from the electrode includes a contribution that depends at least in part on capacitive charging of a surface of the electrode;
fitting the signal to a model of an expected signal to isolate the contribution; and
determining functionalized detection-surface parameter of the electrode using the isolated contribution.
36. The method of example 35, further comprising detecting application of the biosensor to the user's body based at least in part on the functionalized detection-surface parameter of the electrode.
37. The method of example 35 or example 36, further comprising:
applying a correction factor to signals generated at least in part by the electrode, wherein the correction factor depends at least in part on the functionalized detection-surface parameter of the electrode; or
adjusting the drive signal applied to the electrode based at least in part on the functionalized detection-surface parameter of the electrode.
38. A method, comprising:
measuring a signal output from a sensing element of a biosensor, wherein the signal is output in response to an interrogation signal in a drive signal that is applied to the sensing element, wherein the sensing element is positionable at a detection site within tissue of a user to access a body fluid of the user and is configured to detect presence of an analyte of interest in the body fluid;
fitting the signal to a model of an expected signal to isolate a transient response of the signal output, wherein the transient response is associated with one or more characteristics of a detection site; and
determining the presence of the analyte of interest based at least in part on the transient response.
39. A biosensor device, comprising:
a biosensor having a plurality of microneedles configured to access interstitial fluid in a user's skin, wherein each microneedles of the plurality forms at least part of one or more electrodes configured to generate electrical signals in response to one or more analytes of interest in the interstitial fluid; and
an electronics system operably coupled to the biosensor, wherein the electronics system is configured to receive and process the electrical signals, and wherein the electronic system is configured to perform any of the methods of examples 1-38.
40. The biosensor device of example 39, wherein the electronics system is configured to wirelessly communicate with a computing device to receive updates to excitation signals, perturbations, models, correction factors, or any combination thereof
41. A non-transitory, computer-readable medium storing instructions thereon that, when executed by at least one processor of a biosensor device, cause the at least one processor to perform any of the methods of examples 1-38.

### CONCLUSION

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any material incorporated herein by reference conflicts with the present disclosure, the present disclosure controls. The following commonly assigned U.S. patent applications and U.S. patents are incorporated herein by reference in their entireties:

U.S. Pat. No. 10,173,042, filed Dec. 15, 2016, entitled METHOD OF MANUFACTURING A SENSOR FOR SENSING ANALYTES;

U.S. Pat. No. 10,820,860, filed Jan. 19, 2017, entitled ON-BODY MICROSENSOR FOR BIOMONITORING;

U.S. Pat. No. 10,595,754, filed May 22, 2017, entitled SYSTEM FOR MONITORING BODY CHEMISTRY;

U.S. Patent Application Publication No. 2018/0140235, filed Jan. 22, 2018, entitled SYSTEM FOR MONITORING BODY CHEMISTRY;

U.S. Patent Application Publication No. 2020/0077931, filed Sep. 3, 2019, entitled FORECASTING BLOOD GLUCOSE CONCENTRATION;

U.S. Patent Application Publication No. 2020/0375549, filed May 29, 2020, entitled SYSTEMS FOR BIOMONITORING AND BLOOD GLUCOSE FORECASTING, AND ASSOCIATED METHODS;

U.S. patent application Ser. No. 17/167,795, filed Feb. 4, 2021, entitled FORECASTING AND EXPLAINING USER HEALTH METRICS;

U.S. patent application Ser. No. 17/236,753, filed Apr. 21, 2021, entitled SYSTEMS AND METHODS FOR BIOMONITORING AND PROVIDING PERSONALIZED HEALTHCARE;

Patent application. No. PCT/2021/028445, filed Apr. 21, 2021, entitled SYSTEMS AND METHODS FOR BIOMONITORING AND PROVIDING PERSONALIZED HEALTHCARE;

U.S. patent application Ser. No. 17/338,570, filed Jun. 3, 2021, entitled PREDICTIVE GUIDANCE SYSTEMS FOR PERSONALIZED HEALTH AND SELF-CARE, AND ASSOCIATED METHODS;

U.S. patent application Ser. No. 17/338,586, filed Jun. 3, 2021, entitled SYSTEMS FOR ADAPTIVE HEALTHCARE SUPPORT, BEHAVIORAL INTERVENTION, AND ASSOCIATED METHODS; and

U.S. patent application Ser. No. 17/578,386, filed Jan. 18, 2022, entitled SYSTEMS AND METHODS FOR TRACKING AND CALIBRATING BIOSENSORS.

Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising” “including,” “having,” and “with” are used throughout to mean including at least the recited feature(s) such that any greater number of the same features and/or additional types of other features are not precluded. Moreover, the terms “connect” and “couple” are used interchangeably herein and refer to both direct and indirect connections or couplings. For example, where the context permits, element A “connected” or“coupled” to element B can refer (i) to A directly “connected” or directly “coupled” to B and/or (ii) to A indirectly “connected” or indirectly “coupled” to B.

From the foregoing, it will also be appreciated that various modifications may be made without deviating from the disclosure or the technology. For example, one of ordinary skill in the art will understand that various components of the technology can be further divided into subcomponents, or that various components and functions of the technology may be combined and integrated. In addition, certain aspects of the technology described in the context of particular embodiments may also be combined or eliminated in other embodiments. Furthermore, although advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A method of operating a biosensor device, the method comprising:
applying an excitation signal to a biosensor, wherein the biosensor includes a plurality of electrodes positionable within a user's skin to access interstitial fluid therein, wherein an electrode of the plurality of electrodes is configured to detect a presence of an analyte of interest in the interstitial fluid, wherein applying the excitation signal includes applying the excitation signal to the electrode, and wherein the excitation signal includes a time-varying characteristic configured to perturb a diffusion limited steady state of the biosensor;
measuring a response of the biosensor to the time-varying characteristic, wherein the response of the biosensor includes a first contribution that depends at least in part on capacitive charging of a surface of the electrode and a second contribution that depends at least in part on a diffusion limited process fora faradaic response at the electrode;
separating the first contribution from the second contribution; and
determining, based at least in part on the separated first contribution or the separated second contribution, at least one operational parameter that includes:
(a) one or more system-dependent parameters of the biosensor device,
(b) one or more properties of the interstitial fluid surrounding the electrode,
(c) one or more properties of tissue surrounding the electrode, or
(d) any combination thereof.

2. The method of claim 1, wherein:
the at least one operational parameter includes an effective surface area of the electrode; and
the method further comprises detecting application of the biosensor device to the user's body based at least in part on the effective surface area of the electrode.

3. The method of claim 1, further comprising controlling operation of the biosensor based at least in part on the determined at least one operational parameter to increase detection accuracy of the biosensor for the analyte of interest.

4. The method of claim 1, further comprising:
periodically applying the time-varying characteristic to the electrode and monitoring the response of the biosensor; and
adjusting operation of the biosensor based at least in part on changes detected in the response of the biosensor over time.

5. The method of claim 4, wherein the changes correspond to changes at a detection site of the electrode within the user's skin.

6. The method of claim 4, wherein adjusting the operation of the biosensor includes:
performing drift correction or calibration based at least in part on the detected changes;
adjusting the operation of the biosensor according to the drift correction or the calibration to generate a detection output for the analyte of interest; and
determining a concentration of the analyte of interest based on the detection output.

7. The method of claim 1, wherein the response of the biosensor is a current response of the biosensor.

8. The method of claim 1, wherein the time-varying characteristic includes a positive voltage step in the excitation signal.

9. The method of claim 1, wherein:
the excitation signal includes a square wave, a triangular wave, a sawtooth wave, or any combination thereof; and
the time-varying characteristic includes all or a portion of the square wave, the triangular wave, the sawtooth wave, or any combination thereof.

10. (canceled)

11. The method of claim 1, wherein separating the first contribution from the second contribution includes:
fitting the response of the biosensor to a model of an expected response of the biosensor to the time-varying characteristic; and
simultaneously solving for (a) the first contribution and the second contribution or (b) the first contribution, the second contribution, and the third contribution.

12. (canceled)

13. The method of claim 11, wherein the model of the expected response is a model of an expected current response of the biosensor to the time-varying characteristic as measured downstream from an integrated analog filter.

14. The method of claim 11, further comprising modeling the expected response of the biosensor to the time-varying characteristic.

15. The method of claim 1, wherein:
the one or more system-dependent parameters includes an effective surface area of the electrode; and
the determining includes determining the effective surface area from the separated first contribution.

16. The method of claim 15, further comprising determining an absolute concentration of the analyte of interest based at least in part on the effective surface area of the electrode.

17. The method of claim 1, wherein:
the time-varying characteristic is a first time-varying characteristic, and the response is a first response;
the method further comprises monitoring the at least one operational parameter over time; and
the monitoring includes:
redetermining, based at least in part on a second response of the biosensor to a second time-varying characteristic in the excitation signal, the at least one operational parameter; and
comparing the at least one operational parameter determined from the second response to the at least one operational parameter determined from the first response.

18. The method of claim 17, wherein:
the at least one operational parameter determined from the first response include a first effective surface area of the electrode;
the at least one operational parameter determined from the second response include a second effective surface area of the electrode; and
the monitoring further includes determining a position of the electrode within the user's skin has changed based at least in part on a difference between the first effective surface area and the second effective surface area.

19. The method of claim 17, wherein:
the at least one operational parameter determined from the first response include first diffusion properties at a membrane proximate the electrode;
the at least one operational parameter determined from the second response include second diffusion properties at the membrane; and
the monitoring further includes determining a hydration state of the membrane based at least in part on a difference between the first diffusion properties and the second diffusion properties.

20. The method of claim 17, wherein:
the at least one operational parameter determined from the first response includes first diffusion properties of the interstitial fluid and/or the tissue;
the at least one operational parameter determined from the second response includes second diffusion properties of the interstitial fluid and/or the tissue; and
the monitoring further includes determining based at least in part on a difference between the first diffusion properties and the second diffusion properties, (a) an extent of healing of the tissue surrounding the electrode, (b) a physiology of the user has changed, (c) a hydration level of the user has changes, or (d) any combination thereof.

21-23. (canceled)

24. The method of claim 1, wherein:
the excitation signal corresponds to the analyte of interest;
the electrode is further configured to detect presence of another analyte of interest in the interstitial fluid;
the method further comprises applying another excitation signal to the biosensor; and
the other excitation signal corresponds to the other analyte of interest and includes a different time-varying characteristic configured to perturb the diffusion limited steady state of the biosensor.

25. The method of claim 1, wherein:
the electrode is a first electrode;
the analyte is a first analyte of interest;
the excitation signal is a first excitation signal and corresponds to the first analyte of interest;
the method further comprises applying a second excitation signal to a second electrode of the plurality of electrodes;
the second electrode is configured to detect a presence of a second analyte of interest in the interstitial fluid; and
the second excitation signal corresponds to the second analyte of interest and includes a second time-varying characteristic configured to perturb the diffusion limited steady state of the biosensor.

26. (canceled)

27. (canceled)

28. A method, comprising:
determining an effective surface area of an electrode of a biosensor,
wherein the biosensor is configured to determine a concentration of an analyte of interest in interstitial fluid of a user,
wherein the electrode of the biosensor is positionable within tissue of the user to access the interstitial fluid when the biosensor is applied to the user's body, and
wherein determining the effective surface area of the electrode includes determining the effective surface area from capacitive charging of a surface of the electrode that is measured when a perturbation in an excitation signal is applied to the electrode; and
detecting application of the biosensor to the user's body based at least in part on the determined effective surface area of the electrode.

29. The method of claim 28, further comprising determining whether a minimum application threshold is met based at least in part on the determined effective surface area of the electrode.

30. (canceled)

31. (canceled)

32. The method of claim 29, further comprising instructing the user to reapply the biosensor to the user's body based at least in part on a determination that the minimum application threshold is not met.

33. The method of claim 28, further comprising applying a correction factor to signals generated at least in part by the electrode, wherein the correction factor is based at least in part on the determined effective surface area of the electrode.

34. The method of claim 28, further comprising adjusting the excitation signal based at least in part on the determined effective surface area of the electrode.

35-37. (canceled)

38. A method, comprising:
measuring a signal output from a sensing element of a biosensor, wherein the signal is output in response to an interrogation signal in a drive signal that is applied to the sensing element, wherein the sensing element is positionable at a detection site within tissue of a user to access a body fluid of the user and is configured to detect presence of an analyte of interest in the body fluid;
fitting the signal to a model of an expected signal to isolate a transient response of the signal output, wherein the transient response is associated with one or more characteristics of a detection site; and
determining the presence of the analyte of interest based at least in part on the transient response.

39-41. (canceled)

42. The method of claim 38, further comprising determining an effective surface area of the sensing element based at least in part on the isolated transient response.

43. The method of claim 42, further comprising detecting application of the biosensor to the user's body based at least in part on the determined effective surface area.

44. The method of claim 42, further comprising:
applying a correction factor to signals generated at least in part by the sensing element, wherein the correction factor depends at least in part on the determined effective surface area; or
adjusting the drive signal based at least in part on the determined effective surface area.
